SEQUENCE LISTING (39 sequences)

SEQ ID NO: 1 - protein, 6 aa, synthetic construct
  VARIANT 1: note = optionally absent
CKKKTG

SEQ ID NO: 2 - protein, 6 aa, synthetic construct
  VARIANT 6: note = optionally absent
GTKKKC

SEQ ID NO: 3 - protein, 5 aa, synthetic construct
GTKKK

SEQ ID NO: 4 - protein, 5 aa, synthetic construct
KKKTG

SEQ ID NO: 5 - protein, 8 aa, synthetic construct
  MOD_RES 1: note = X is pSerine4
XGGSGGGS

SEQ ID NO: 6 - protein, 8 aa, synthetic construct
  MOD_RES 8: note = X is pSerine4
GGSGGGSX

SEQ ID NO: 7 - protein, 13 aa, synthetic construct
  VARIANT 5: note = 5' linkage to the antigen
  MOD_RES 13: note = X is pSerine4
GTKKKGGSGGGSX

SEQ ID NO: 8 - protein, 13 aa, synthetic construct
  VARIANT 8: note = 5' linker to antigen
  MOD_RES 1: note = X is pSerine4
XGGSGGGSKKKTG

SEQ ID NO: 9 - protein, 13 aa, synthetic construct
  VARIANT 5: note = 5' maleimide
  MOD_RES 13: note = X is pSerine4
GTKKKGGSGGGSX

SEQ ID NO: 10 - protein, 13 aa, synthetic construct
  MOD_RES 1: note = X is pSerine4
  VARIANT 8: note = 5' maleimide
XGGSGGGSKKKTG

SEQ ID NO: 11 - protein, 637 aa, synthetic construct
ETGAENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 12 - protein, 634 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNYAPKLRSMMRGEIKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPSNNTVKSIRIGPGQAFYYFGDVLGHVRMAHCNISKATWNETLGKVVKQLRKHFGNNTIIRFAQSSGGDLEVTTHSFNCGGEFFYCNTSGLFNSTWISNTSVQGSNSTGSNDSLILPCWIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 13 - protein, 634 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNYAPKLLSNMRGEIKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYFGDVLGHVRMAHCNISKATWNETLGKVVKQLRKHFGNNTIIRFAQSSGGDLEVTTHSFNCGGEFFYCNTSGLFNSTWISNTSVQGSNSTGSNDSLILPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 14 - protein, 634 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNYAPKLRSMMRGEIKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYFGDVLGDVRMAHCNISKATWNETLGKVVKQLRKHFGNNTIIRFAQSSGGDLEVTTHSFNCGGEFFYCNTSGLFNSTWISNTSVQGSNSTGSNDSLILPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 15 - protein, 528 aa, synthetic construct
CIGYHANNSTDTVDTVLEKNVTVTHSVNLLEDSHNGKLCLLKGIAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPQRETRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGGSGYIPEAPRDGQAYVRKDGEWVLLSTFLG

SEQ ID NO: 16 - protein, 14 aa, synthetic construct
  VARIANT 6: note = 5' linker to antigen
  MOD_RES 14: note = X is pSerine4
CGTKKKGGSGGGSX

SEQ ID NO: 17 - protein, 14 aa, synthetic construct
  MOD_RES 1: note = X is pSerine4
  VARIANT 8: note = 5' linker to antigen
XGGSGGGSKKKTGC

SEQ ID NO: 18 - protein, 14 aa, synthetic construct
  VARIANT 6: note = 5' maleimide
  MOD_RES 14: note = X is pSerine4
CGTKKKGGSGGGSX

SEQ ID NO: 19 - protein, 14 aa, synthetic construct
  MOD_RES 1: note = X is pSerine4
  VARIANT 8: note = 5' maleimide
XGGSGGGSKKKTGC

SEQ ID NO: 20 - protein, 7 aa, synthetic construct
GGSGGGS

SEQ ID NO: 21 - protein, 647 aa, synthetic construct
ETGAENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGTKHHHHHHC

SEQ ID NO: 22 - protein, 8 aa, synthetic construct
  VARIANT 1: note = 5' maleimide
  VARIANT 8: note = X is pSer4
GGSGGGSX

SEQ ID NO: 23 - protein, 10 aa, synthetic construct
GTKGHGHGHC

SEQ ID NO: 24 - protein, 8 aa, synthetic construct
GTKHHHHC

SEQ ID NO: 25 - protein, 8 aa, synthetic construct
GTKGHGKC

SEQ ID NO: 26 - protein, 8 aa, synthetic construct
GTKKKKKC

SEQ ID NO: 27 - protein, 7 aa, synthetic construct
GTKHHHC

SEQ ID NO: 28 - protein, 7 aa, synthetic construct
GTKKKKC

SEQ ID NO: 29 - protein, 6 aa, synthetic construct
GTKHHC

SEQ ID NO: 30 - protein, 6 aa, synthetic construct
GTKKKC

SEQ ID NO: 31 - protein, 7 aa, synthetic construct
GTKEEEC

SEQ ID NO: 32 - protein, 9 aa, synthetic construct
GTKGHGHGH

SEQ ID NO: 33 - protein, 7 aa, synthetic construct
GTKHHHH

SEQ ID NO: 34 - protein, 7 aa, synthetic construct
GTKGHGK

SEQ ID NO: 35 - protein, 7 aa, synthetic construct
GTKKKKK

SEQ ID NO: 36 - protein, 6 aa, synthetic construct
GTKHHH

SEQ ID NO: 37 - protein, 6 aa, synthetic construct
GTKKKK

SEQ ID NO: 38 - protein, 5 aa, synthetic construct
GTKHH

SEQ ID NO: 39 - protein, 6 aa, synthetic construct
GTKEEE

(19) United States
(12) Patent Application Publication
(10) Pub. No.: US 2026/0295039 A1
(43) Pub. Date: Oct. 1, 2026

(54) IMPROVED ALUM-ANCHORED IMMUNOGENS

(71) Applicants: Massachusetts Institute of Technology, Cambridge, MA (US); THE SCRIPPS RESEARCH INSTITUE, La Jolla, CA (US)

(72) Inventors: Darrell IRVINE, Cambridge, MA (US); Kristen Alexandra RODRIGUES, Cambridge, MA (US); William R. SCHIEF, La Jolla, MA (US)

(21) Appl. No.: 19/480,501

(22) PCT Filed: Apr. 30, 2024
(86) PCT No.: PCT/US2024/026964

§ 371 Date: Oct. 31, 2025

Related U.S. Application Data
(60) Provisional application No. 63/500,068, filed on May 4, 2023.

(51) Int. Cl. A61K 39/39; A61K 39/00; A61K 39/145; A61K 39/21; A61K 39/385; A61P 31/18; A61P 37/04; C07K 14/005

(52) U.S. Cl. CPC A61K 39/39; A61K 39/145; A61K 39/21; A61K 39/385; A61P 31/18; A61P 37/04; C07K 14/005; A61K 2039/55505; A61K 2039/55555; A61K 2039/55577; C07K 2319/00; C12N 2740/16134; C12N 2760/16034

(57) ABSTRACT

Compositions are provided, of the general formula X1-X2-X3, wherein (a) X2 comprises an antigen; (b) X1 is absent or comprises or consists of the amino acid sequence (C)KKKTG (SEQ ID NO: 1); and (c) X3 is absent or comprises or consists of the amino acid sequence GTKKK(C) (SEQ ID NO:2), wherein (i) only one of X1 and X3 is present, and (ii) the residue m parentheses m SEQ ID NO: 1 and SEQ ID NO: 2 is optional and may be present or may be deleted.

### FEDERAL FUNDING STATEMENT

This invention was made with government support under W911NF-18-2-0048 awarded by the U.S. Army Research Office, and A1100663, AI161818, AI161297, AI125068, AI144462, AI048240 awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Mar. 28, 2024 having the file name “23-0655-WO.xml” and is 46,316 bytes in size.

### BACKGROUND

The human Immunodeficiency virus/acquired immnunodeficiency syndrome (IV/AIDS) epidemic began more than 40 years ago. Despite the increasing availability of antiretroviral drugs, HIV remains a leading cause of death globally, with over 37 million people currently infected with HIV worldwide. As a result, there is a significant unmet need for the development of an effective prophylactic HIV vaccine as a low-cost, facile solution to prevent infection.

### SUMMARY

In one aspect, the disclosure provides a composition comprising the general formula X1-X2-X3, wherein
(a) X2 comprises a antigen;
(b) X1 is absent or comprises or consists of the amino acid sequence (C)KKKTG (SEQ ID NO:1); and
(c) X3 is absent or comprises or consists of the amino acid sequence GTKKK(C) (SEQ ID NO:2),
wherein (i) only one of X1 and X3 is present, and (ii) the residue in parentheses in SEQ ID NO: 1 and SEQ ID NO:2 is optional and may be present or may be deleted.

In one embodiment, the antigen comprises a polypeptide antigen, and the composition comprises a fusion protein. In another embodiment, optional amino acid linkers may be present between the X1-X2 or X2-X3. In a further embodiment, X2 is present and consists of GTKKK(C) (SEQ ID NO:2) or GTKKK (SEQ ID NO:3). In one embodiment, X1 is present and consists of the amino acid sequence (C)KKKTG (SEQ ID NO:1) or KKKTG (SEQ ID NO:4). In another embodiment, the composition or fusion protein comprises the formula B1-X1-X2 or X2-X3-B2, wherein
B1 comprises pSer4-GGSGGGS-Z1 (SEQ ID NO:5), or
B2 comprises Z1-GGSGGGS-pSer4 (SEQ ID NO:6);
wherein Z1 comprises a linkage to the antigen.

In another embodiment, Z1 is selected from the group consisting of maleimide, 5-hydroxy-pyrrolone, a 3-arylpropionitrile, and methylsulfonyl phenyloxadiazole. In a further embodiment, the composition or fusion protein comprises:

(SEQ ID NO: 7)
X2-GTKKK-Z1-GGSGGGS-pSer4;
or
(SEQ ID NO: 8)
pSer4-GGSGGGS-Z1-KKKTG-X2,
or
(SEQ ID NO: 9)
X2-GTKKK-maleimide-GGSGGGS-pSer4;
or
(SEQ ID NO: 10)
pSer4-GGSGGGS-maelimide-KKKTG-X2.

In some embodiments, the antigen comprises an antigen from a pathogen, a tumor antigen, a viral antigen or a bacterial antigen. In one embodiment, the antigen comprises a human immunodeficiency virus (HIV) envelope (Env) antigen, an influenza hemagglutinin (HLA) antigen, or an influenza neuraminidase (NA) antigen. In another embodiment, the antigen comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO:11-15, or an antigenic fragment thereof.

In another aspect, the disclosure provides a polypeptide comprising a sequence selected from the group consisting of CGTKKK-Z1-GGSGGGS-pSer4 (SEQ ID NO:16), pSer4-GGSGGGS-Z1-KKKTGC (SEQ ID NO:17), CGTKKK-maleimide-GGSGGGS-pSer4 (SEQ ID NO: 18) and pSer4-GGSGGGS-maleimide-KKKTGC (SEQ ID NO:19) wherein Z1 is a linkage. In one embodiment, the composition, fusion protein, or polypeptide is bound to aluminum hydroxide via pSer4. In another embodiment, the disclosure provides a formulation, comprising the composition, fusion protein, or polypeptide bound to aluminum hydroxide via pSer4, and further comprising a non-liposome, non-micelle particle, wherein the particle comprises a lipid, a sterol, a saponin, and an optional additional non-alum adjuvant, wherein the particle is optionally bound to the alum. In one embodiment, the particle is a porous, cage-like nanoparticle about 30 nm to about 60 nm in diameter. In various embodiments the lipid is a phospholipid, the sterol comprises cholesterol or a derivative thereof, and/or the saponin is a natural or synthetic saponin.

In various embodiments, the additional adjuvant is present and comprises a toll-like receptor (TLR)4 agonist, a pathogen-associated molecular pattern (PAMP), and/or a TLR ligand.

The disclosure further provides pharmaceutical compositions comprising the composition, fusion protein, polypeptide, or formulation of any embodiment, and a pharmaceutically acceptable carrier. In another embodiment, the disclosure comprises a vaccine comprising the composition, fusion protein, polypeptide, or formulation. In other aspects, the disclosure provides a method for generating an immune response against an antigen, comprising administering to a subject an amount effective to generate an immune response in the subject of the composition, fusion protein, polypeptide, formulation, or vaccine of any embodiment; and a method of treating a subject in need thereof comprising administering to the subject the composition, fusion protein, polypeptide, formulation, or vaccine of any embodiment in an effective amount to induce an immune response against the antigen.

### DESCRIPTION OF THE FIGURES

FIG. 1 Combining MD39-pSer with alum-binding saponin nanoparticle co-adjuvant enhances humoral responses, a Schematic overview of parameters modified for pSer-tagged trimers, including phosphoserine valency, immunogen linker tag sequence, pSer spacing, flexible linker spacer sequence, and effects of adding co-adjuvants to alum. b BALB/c mice (n=5-11 animals/group) were immunized as indicated with varying amounts of fluorescently labeled antigen, and the fluorescence at the injection site was quantified longitudinally. Values plotted are means±standard deviation. Statistical significance was determined by two-way ANOVA followed by Tukey's post-hoc test. c Mice (n=5 animals/group) were immunized as indicated, and serum IgG responses were assessed longitudinally by ELISA. Arrows indicate immunizations. Values plotted are geometric means±geometric standard deviation. Statistical significance was determined by two-way ANOVA followed by Tukey's post-hoc test. d Mice were immunized as indicated, and germinal center (GC) B cell responses in draining inguinal lymph nodes were analyzed by flow cytometry 14 days post-immunization, e Shown are representative flow cytometry gating plots of MD39-specific GC B cell analysis, plotted in f. Values plotted are means standard deviation. Statistical significance was determined by one-way ANOVA followed by Tukey's post-hoc test. ns p>0.05, *p<0.05, **p<0.01, ***p<0.001, ****p<0.0001.

FIG. 2. Phosphoserine valency modulates immunogen binding to alum and humoral responses to immunization. a MD39 conjugated to peptides containing 4 or 8 phosphoserines (MD39-pSer4, MD39-pSer₈) were assayed for phosphates by a malachite green assay. b Antigenicity profiling of MD39-pSer4 and MD39-pSer₈ on alum compared to unmodified MD39 captured by anti-histag. Shown are area-under-the-curve values for trimer binding vs. antibody concentration. c pSer-conjugated, Ser-conjugated or unmodified MD39 trimers were mixed with alum, and the fraction of protein bound to alum was assessed after 24-hour incubation in varying percentages of mouse serum at 37° C. d BALB/c mice (n=3 animals/group) were immunized with 10 μg fluorescently labeled MD39 plus alum and injection site fluorescence was quantified longitudinally. e Mice (n=3-10 animals/group) were immunized with 10 μg unmodified, pSer₄-, or pSer₈-conjugated MD39 and 100 μg alum, and antigen-specific germinal center (GC) B cell responses in draining inguinal lymph nodes were analyzed by flow cytometry 14 days post-immunization. f-g Shown are MD39-specific GC B cell counts and staining plots. h BALB/c mice (n=5 animals/group) were immunized with 5 μg unmodified, pSer₄- or pSer₈-conjugated MD39 and 50 μg alum plus 5 μg SMNP, and serum IgG responses were assessed by ELISA. Statistical significance was determined by Mann-Whitney test for a, one-way ANOVA for e, and two-way ANOVA for b, c, d, and h followed by Tukey's post-hoc test. Values plotted are means±standard deviation. ns p>0.05, *p<0.05, **p<0.01, ***p<0.001, ****p<0.0001.

FIG. 3. MD39 constructs containing alternate linkers retain alum binding properties and antigenicity profile when conjugated to pSer. a Table of alternate linker sequences and their attributes (from top to bottom SEQ ID NOs: 23-31 b MD39 constructs containing these linkers were expressed, conjugated to pSer₄ peptides, and assayed for phosphates per protein by a malachite green assay. Values plotted are means±standard deviation. c pSer-conjugated or unmodified MD39 constructs were mixed with alum, and the fraction of protein bound to alum was assessed before (“Loading”) and after incubation for 24 hours in 10% mouse serum at 37° C. Values plotted are means±standard deviation. Statistical significance was determined by two-way ANOVA followed by Sidak's post-hoc test. d Antigenicity profiling of MD39-pSer₄ on alum compared to MD39_his-pSer₄. Shown are the area under individual binding curves. Values plotted are means±standard deviation. Statistical significance was determined by two-way ANOVA followed by Sidak's post-hoc test. ns p>0.05, *p<0.05, **p<0.01, ***p<0.01, ****p<0.0001.

FIG. 4. MD39 constructs containing alternate linkers conjugated to pSer elicit strong humoral immune responses. a BALB/c mice (n=5 animals/group) were immunized with 10 μg Ser₄- or pSer₄-conjugated MD39 constructs and 100 μg alum and boosted with 5 μg Ser₄- or pSer₄-conjugated MD39 constructs and 50 μg alum with 5 μg SMNP at 6 weeks. Serum IgG responses were assessed longitudinally by ELISA. Arrows indicate immunizations. b Individual responses plotted for day 28. c Individual responses plotted for day 42. d Individual responses plotted for day 56. Values plotted are geometric means±geometric standard deviation. Statistical significance was determined by two-way ANOVA followed by Tukey's post-hoc test. e Mice (n=5 animals/group) were immunized with 5 μg pSer₄- or Ser₄-conjugated MD39_nohis8 construct and 50 μg alum with 5 μg SMNP, and germinal center (GC) B cell responses were assessed in the draining inguinal lymph nodes at day 14 and 21 post-immunization. f Shown are representative flow cytometry gating plots of MD39-specific GC B cell analysis, plotted in g. Statistical significance was determined by two-way ANOVA followed by Tukey's post-hoc test, Values plotted are means±standard deviation. ns p>0.05, *p<0.05, **p<0.01, ***p<0.001, ****p<0.0001.

FIG. 5. Glycine spacers between pSer residues do not significantly alter the physical properties of MD39-pSer. a MD39 was conjugated to pSer linkers containing 0-3 glycine residue spacers between pSer residues, and these constructs were mixed with alum. The fraction of protein bound to alum was assessed before (“Loading”) and after incubation for 24 hours in varying amounts of mouse serum at 37° C. Values plotted are means±standard deviation. Statistical significance was determined by two-way ANOVA followed by Tukey's post-hoc test. b-c Antigenicity profiling of MD39-pSer₄ constructs, Shown are the area under individual binding curves. Values plotted are means±standard deviation. Statistical significance was determined by two-way ANOVA followed by Tukey's post-hoc test. ns p>0.05, *p<0.05, **p<0.01, ***p<0.001, ****p<00001.

FIG. 6. Replacing the 6-unit PEG spacer with a flexible glycine/serine spacer does not significantly alter the physical properties of MD39_nohis8_congly-pSer. Chemical structures of pSer linkers containing (a) 6-unit poly(ethylene glycol) (PEG₆) or (b) glycine-glycine-serine-glycine-glycine-glycine-serine repeat (GGSGGGS (SEQ ID NO: 20)) spacers. c pSer-conjugated MD39 constructs were mixed with alum, and the fraction of protein bound to alum was assessed before and after 24-hour incubation in varying percentages of mouse serum at 37° C. d Antigenicity profiling of MD39_nohis8_congly-pSer₄ captured on alum. Shown are the area-under-individual binding curves. e BALB/c mice were immunized with 5 μg pSer₄-conjugated MD39 constructs and 50 μg alum with 5 μg SMNP, and germinal center (GC) B cell responses in draining inguinal lymph nodes were analyzed by flow cytometry 14 days post-immunization. f Shown are representative flow cytometry gating plots of MD39-specific GC B cell analysis, plotted by MD39-specific GC B cell count in g. h Mice (n=5 animals/group) were immunized with 10 μg pSer₄-conjugated MD39 and 100 μg alum and boosted with 5 μg Ser₄- or pSer₄-conjugated MD39 and 50 μg alum with 5 μg SMNP at 6 weeks, and serum IgG responses were assessed longitudinally by ELISA. Arrows indicate immunizations. Values plotted are means±standard deviation in c-f, and geometric means±geometric standard deviation in h. Statistical significance was determined by two-way ANOVA followed by Sidak's post-hoc test in c, d, and h, and one-way ANOVA followed by Tukey's post-hoc test in e and g. ns p>0.05, *p<0.05, **p<0.01, ***p<0.001, ****p<0.0001.

FIG. 7. SMNP exhibits strong binding to alum. a Saponin/phospholipid nanoparticle adjuvant (SMNP) was mixed with alum with or without MD39-pSer₄, and the fraction of SMNP bound to alum was assessed after incubation for 24 hours in 10% mouse serum at 37° C. Values plotted are means±standard deviation. Statistical significance was determined by one-way ANOVA followed by Tukey's post-hoc test. ns p>0.05.

FIG. 8. Phosphoserine-conjugated MD39 retains the physical properties of MD39. a Chemical structure of phosphoserine (pSer) peptide containing a maleimide group linked to a 6-unit poly(ethylene glycol) spacer followed by repeat pSer residues. Thiol-maleimide coupling mediates pSer-conjugation of MD39 trimers. b Antigenicity profiling of MD39-pSer₄ and MD39-pSer₈ captured on alum. Shown are binding profiles of antibodies (n=3 replicates). Values plotted are means±standard deviation. c Representative flow cytometry gating approach for MD39-specific germinal center (GC) B cells.

FIG. 9. Phosphoserine-conjugated MD39 constructs with alternate linkers exhibit strong alum binding and retain the antigenicity profile of MD39. a pSer-conjugated MD39 constructs were mixed with alum, and the fraction of protein bound to alum was assessed before (“Loading”) and after incubation for 24 hours in varying percentages of mouse serum at 37° C. Values plotted are means±standard deviation, Statistical significance was determined by two-way ANOVA followed by Sidak's post-hoc test. b Antigenicity profiling of MD39-pSer₄ captured on alum (n=3 replicates). Values plotted are means±standard deviation. ns p>0.05, ****p<0.0001.

FIG. 10. pSer-conjugated MD39 constructs with alternate linkers elicit strong humoral immune responses. BALB/c mice (n=5 animals/group) were immunized with 10 μg Ser₄- or pSer₄-conjugated MD39 constructs and 100 μg alum and boosted with 5 μg Ser₄-or pSer₄-conjugated MD39 constructs and 50 μg alum with 5 μg SMNP at 6 weeks, and serum IgG responses were assessed by ELISA at day 56. a Shown are absorbance curves for MD39_his-Ser₄ immunized mice measured against MD39 and MD39 base-blocked with 12N antibody. b Plotted are the fraction of integrated absorbance curves retained in ELISAs completed in the presence of base-blocking 12N antibody. Dashed line indicates value for which there is no MD39 base-directed response. Values plotted are means±standard deviation. Statistical significance was determined by one-way ANOVA followed by Tukey's post-hoc test. ns p>0.05, *p<0.05.

FIG. 11. Glycine spacers between pSer residues do not significantly alter the antigenicity profile of MD39-pSer₄. a Chemical structures of pSer linkers containing 0-3 glycine spacers (pS₄, (pSG)₄, (pSGG)₄, and (pSGG)₄, respectively). b Antigenicity profiling of unmodified or pSer-conjugated MD39_nohis8 (n:=3 replicates). Values plotted are means±standard deviation.

FIG. 12. Glycan hole filled MD39 exhibits strong alum binding and retains antigenicity profile when conjugated to pSer₄. a pSer-conjugated or unmodified MD39 constructs were mixed with alum, and the fraction of protein bound to alum was assessed before (“Loading”) and after incubation for 24 hours in 10% mouse serum at 37° C. Values plotted are means±standard deviation. Statistical significance was determined by one-way ANOVA followed by Tukey's post-hoc test. b Antigenicity profiling of MD39-pSer₄ with and without a filled glycan hole (congly) on alum compared to unmodified MD39 captured by mVRC01. Values plotted are means±standard deviation. c Antigenicity profiling of unmodified or pSer-conjugated MD39 with and without a filled glycan hole (n=3 replicates). Shown are the area under individual binding, curves. Statistical significance was determined by two-way ANOVA followed by Tukey's post-hoc test. ns p>0.05, *p<0.05, ****p<00001.

FIG. 13. MD39-pSer construct with a filled glycan hole (congly) elicits strong humoral immune responses. a BALB/c mice were immunized with 5 μg pSer₄-conjugated MD39 constructs and 50 μg alum with 5 μg SMNP, and germinal center (GC) B cell responses in draining inguinal lymph nodes were analyzed by flow cytometry 14 days post-immunization. Values plotted are means±standard deviation. Statistical significance was determined by one-way ANOVA followed by Tukey's post-hoc test. b Shown are representative flow cytometry gating plots of MD39-specific GC B cell analysis, plotted by MD39-specific GC B cell count in c and the percentage of GC B cells that are MD39-specific in d. Values plotted are means±standard deviation. Statistical significance was determined by one-way ANOVA followed by Tukey's post-hoc test. e Mice (n=5 animals/group) were immunized with 10 μg pSer₄-conjugated MD39 constructs and 100 μg alum and boosted with 5 μg Ser₄- or pSer₄-conjugated MD39 constructs and 50 μg alum with 5 μg SMNP at 6 weeks, and serum IgG responses were assessed longitudinally by ELISA. Arrows indicate immunizations. Values plotted are geometric means±geometric standard deviation. Statistical significance was determined by two-way ANOVA followed by Sidak's post-hoc test. ns p>0.05, *p<0.05, **p<001, ***p<0,001, ****p<0.0001.

FIG. 14. Replacing the 6-unit PEG spacer with a flexible glycine/serine spacer does not significantly alter the antigenicity profile of MD39-pSer. a Antigenicity profiling of MD39-pSer₄ with either PEG₆ or GGSGGGS (SEQ ID NO. 20) spacer (n=3 replicates). b Mice (n=5 animals/group) were immunized with 10 μg pSer-conjugated MD39 and 100 μg alum and boosted with 5 μg pSer₄-conjugated MD39 and 50 μg alum with 5 μg SMNP at 6 weeks, and anti-PEG serum IgM responses were assessed at week 8 by ELISA. Shown are individual mouse ELISA curves, which are calculated as area under the curve (AUC) in c. Values plotted are means±=standard deviation. Statistical significance was determined by unpaired t test. ns p>0.05, **p<0.01.

FIG. 15. pSer-modification of N332-GT5 immunogen facilitates binding to alum with retention of key structural epitopes. (a) N332-GT5 conjugated to pSer₄ was assayed for phosphates by a malachite green assay. (b) N332-GT5 and pSer-conjugated N332-GT5 were mixed with alum, and the fraction of protein bound to alum was assessed before (“Loading”) and after incubation for 24 hours in varying percentages of mouse serum at 37° C. Statistical significance was determined by two-way NOVA followed by Sidak's post-test. (c) Antigenicity profiling of pSer-modified N332-GT5. Values plotted are means±standard deviation. n.s. p>0.05, *p<0.05, **p<0.01, ***p<0.001, ****p<0.0001.

### DETAILED DESCRIPTION

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise. throughout the description and the claims, the words ‘comprise’, ‘comprising’, and the like are to be construed in an inclusive sense as opposed to an exclusive or exhaustive sense; that is to say, in the sense of “Including, but not limited to”. Words using the singular or plural number also include the plural or singular number, respectively. Additionally, the words “herein,” “above” and “below” and words of similar import, when used in this application, shall refer to this application as a whole and not to any particular portions of this application.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), gly cine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

In all embodiments of polypeptides disclosed herein, any N-terminal methionine residues are optional (i.e.: the N-terminal methionine residue may be present or may be absent, and may be included or excluded when determining percent amino acid sequence identity compared to another polypeptide).

As used herein, the term “nanoparticle” refers to submicron particles less 100 nm in dimension. In some embodiments, when nanoparticles form aggregates, the size of the aggregates may exceed 100 nm.

As used herein, “about” will mean up to plus or minus 5% of the particular value.

As used herein, the term “adjuvant” refers to any substance that acts to augment and/or direct antigen-specific immune responses when used in combination with specific antigens. When combined with a vaccine antigen, adjuvant increases the immune response to the vaccine antigen as compared to the response induced by the vaccine antigen alone. Adjuvants help drive immunological mechanisms and shape the output immune response to vaccine antigens.

In one aspect, the disclosure provides compositions comprising the general formula X1-X2-X3, wherein
(a) X2 comprises an antigen;
(b) X1 is absent or comprises or consists of the amino acid sequence (C)KKKTG (SEQ ID NO:1); and
(c) X3 is absent or comprises or consists of the amino acid sequence GTKKK(C) (SEQ ID NO:2),

wherein (i) only one of X1 and X3 is present, and (ii) the residue in parentheses in SEQ ID NO:1 and SEQ ID NO:2 is optional and may be present or may be deleted.

As disclosed in the accompanying examples, the inventors have demonstrated that the composition can be used as a key component for coupling an antigen to alum (aluminum hydroxide) with surprisingly strong binding affinity while improving immunogenicity of the linked antigen. Thus, the compositions according to this aspect of the disclosure are especially effective for generating an immune response against the antigen component of the composition.

In one embodiment, optional amino acid linkers may be present between the X1-X2 or X2-X3. In this embodiment, the amino acid linkers may be of any length or amino acid content as suitable for an intended use. In some embodiments, a linker may be used when accessibility to epitopes in the antigen closer to X1 or X3 are desired.

In one embodiment, wherein X2 is present and consists of GTKKK(C) (SEQ ID NO:2) or GTKKK (SEQ ID NO:3). In another embodiment, X1 is present and consists of the amino acid sequence (C)KKKTG (SEQ ID NO: 1) or KKKTG (SEQ ID NO:4).

In another embodiment, the composition comprises the formula B1-X1-X2 or X2-X3-B2, wherein
B1 comprises pSer4-GGSGGGS-Z1 (SEQ ID NO:5), or
B2 comprises Z1-GGSGGGS-pSer4 (SEQ ID NO:6);
wherein Z1 comprises a linker to the antigen.

Z1 may comprise any suitable linkage to the antigen. Z1 may comprise or consist of any moiety that can link X1 or X2 to the antigen via any suitable bio conjugation chemistries, including but not limited to click chemistry groups (azide/alkyne), streptavidin-tags, sortase, or any other bio-orthogonal chemistry. In various non-limiting embodiments, Z1 may be maleimide, 5-hydroxy-pyrrolone, a 3-arylpropionitrile, or methylsulfonyl phenyloxadiazole.

In one embodiment, the composition or fusion protein comprises:

(SEQ ID NO: 7)
X2-GTKKK-Z1-GGSGGGS-pSer4;
or
(SEQ ID NO: 8)
pSer4-GGSGGGS-Z1-KKKTG-X2.

In another embodiment, the composition or fusion protein comprises:

(SEQ ID NO: 9)
X2-GTKKK-maleimide-GGSGGGS-pSer4;
or
(SEQ ID NO: 10)
pSer4-GGSGGGS-maelimide-KKKTG-X2.

In these embodiments, the composition or fusion protein is capable of coupling an antigen to alum (aluminum hydroxide) via the pSer4 domain with surprisingly strong binding affinity, and provides for improved immunogenicity.

The composition or fusion protein may comprise any antigen as deemed appropriate for an intended use. The antigen may comprise peptides, proteins, polysaccharides, saccharides, lipids, nucleic acids, or combinations thereof. The antigen may be derived from a virus, bacterium, parasite, plant, protozoan, fungus, tissue or transformed cell such as a cancer or leukemic cell. The antigen may be covalently linked to X1 or X2 via any suitable means.

In one embodiment, the antigen comprises an antigenic polypeptide. In this embodiment, the composition comprises a fusion polypeptide. Any polypeptide antigen may be used, including antigens from a pathogen or a tumor. In one embodiment, the polypeptide antigen comprises a bacterial, viral, fungal, or other parasitic antigen.

In one embodiment, the antigen is a polypeptide antigen that is capable of forming a multimer, including but not limited to a dimer, trimer, tetramer, pentamer, hexamer, etc. In one embodiment, the antigen comprises an influenza neuraminidase (NA) antigen that is capable of forming a tetramer.

In one embodiment, the antigen is a polypeptide antigen that is capable of forming a trimer. In exemplary embodiments, the antigen comprises a human immunodeficiency virus (HIV) envelope (Env) antigen or an influenza hemagglutinin (HA) antigen, each of which are capable of trimer formation. In a further embodiment, the antigen comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO:11-15, or an antigenic fragment thereof.

(SEQ ID NO: 11; MD39)
ETGAENLWVTVYYGVPVWKDAETTLFCASDAKAYETEKHNVWATHACVPTDPNPQEIHLENVTEEENM
WKNNMVEQMHEDIISLWDQSLKPCVKLTPLCVTLQCTNVINNITDDMRGELKNCSFNMTTELRDKKQK
VYSLFYRLDVVQINENQGNRSNNSNKEYRLINCNTSAITQACPKVSFEPIPIHYCAPAGFAILKCKDK
KFNGTGPCPSVSTVQCTHGIKPVVSTQLLINGSLAEEEVIIRSENITNNAKNILVQLNTPVQINCTRP
NNNTVKSIRIGPGQAFYYTGDIIGDIRQAHCNVSKATWNETLGKVVKQLRKHFGNNTIIRFAQSSGGD
LEVTTHSENCGGEFFYCNTSGLENSTWISNTSVQGSNSTGSNDSITLPCRIKQIINMWQRIGQAMYAP
PIQGVIRCVSNITGLILTRDGGSTNSTTETFRPGGGDMRDNWRSELYKYKVVKIEPLGVAPTRCKRRV
VGRRRRRRAVGIGAVSLGFLGAAGSTMGAASMTLTVQARNLLSGIVQQQSNLLRAPEPQQHLLKDTHW
GIKQLQARVLAVEHYLRDQQLLGIWGCSGKLICCTNVPWNSSWSNRNLSEIWDNMTWLQWDKEISNYT
QIIYGLLEESQNQQEKNEQDLLALD
(SEQ ID NO: 12; N332-GT5)
AENLWVTVYYGVPVWKDAETTLFCASDAKAYETEKHNVWATHACVPTDPNPQEIHLENVTEEENMWKN
NMVEQMHEDIISLWDQSLKPCVKLTPLCVTLQCTNYAPKLRSMMRGEIKNCSFNMTTELRDKKQKVYS
LFYRLDVVQINENQGNRSNNSNKEYRLINCNTSAITQACPKVSFEPIPIHYCAPAGFAILKCKDKKEN
GTGPCPSVSTVQCTHGIKPVVSTQLLINGSLAEEEVIIRSENITNNAKNILVQLNTPVQINCTRPSNN
TVKSIRIGPGQAFYYFGDVLGHVRMAHCNISKATWNETLGKVVKQLRKHFGNNTIIRFAQSSGGDLEV
TTHSFNCGGEFFYCNTSGLENSTWISNTSVQGSNSTGSNDSLILPCWIKQIINMWQRIGQAMYAPPIQ
GVIRCVSNITGLILTRDGGSINSTTETFRPGGGDMRDNWRSELYKYKVVKIEPLGVAPTRCKRRVVGR
RRRRRAVGIGAVSLGFLGAAGSTMGAASMTLTVQARNLLSGIVQQQSNLLRAPEPQQHLLKDTHWGIK
QLQARVLAVEHYLRDQQLLGIWGCSGKLICCTNVPWNSSWSNRNLSEIWDNMTWLQWDKEISNYTQII
YGLLEESQNQQEKNEQDLLALD
(SEQ ID NO: 13; N332-GT1)
AENLWVTVYYGVPVWKDAETTLECASDAKAYETEKHNVWATHACVPTDPNPQEIHLENVTEEENMWKN
NMVEQMHEDIISLWDQSLKPCVKLTPLCVTLQCTNYAPKLLSNMRGEIKNCSFNMTTELRDKKQKVYS
LFYRLDVVQINENQGNRSNNSNKEYRLINCNTSAITQACPKVSFEPIPIHYCAPAGFAILKCKDKKEN
GTGPCPSVSTVQCTHGIKPVVSTQLLINGSLAEEEVIIRSENITNNAKNILVQLNTPVQINCTRPNNN
TVKSIRIGPGQAFYYFGDVLGHVRMAHCNISKATWNETLGKVVKQLRKHEGNNTIIRFAQSSGGDLEV
TTHSFNCGGEFFYCNTSGLENSTWISNTSVQGSNSTGSNDSLILPCRIKQIINMWQRIGQAMYAPPIQ
GVIRCVSNITGLILTRDGGSTNSTTETFRPGGGDMRDNWRSELYKYKVVKIEPLGVAPTRCKRRVVGR
RRRRRAVGIGAVSLGFLGAAGSTMGAASMTLTVQARNLLSGIVQQQSNLLRAPEPQQHLLKDTHWGIK
QLQARVLAVEHYLRDQQLLGIWGCSGKLICCINVPWNSSWSNRNLSEIWDNMTWLQWDKEISNYTQII
YGLLEESQNQQEKNEQDLLALD
N332-GT2 sequence:
(SEQ ID NO: 14; N332-GT2)
AENLWVTVYYGVPVWKDAETTLFCASDAKAYETEKHNVWATHACVPTDPNPQEIHLENVTEEFNMWKN
NMVEQMHEDIISLWDQSLKPCVKLTPLCVTLQCTNYAPKLRSMMRGEIKNCSFNMTTELRDKKQKVYS
LFYRLDVVQINENQGNRSNNSNKEYRLINCNTSAITQACPKVSFEPIPIHYCAPAGFAILKCKDKKEN
GTGPCPSVSTVQCTHGIKPVVSTQLLINGSLAEEEVIIRSENITNNAKNILVQLNTPVQINCTRENNN
TVKSIRIGPGQAFYYFGDVLGDVRMAHCNISKATWNETLGKVVKQLRKHEGNNTIIRFAQSSGGDLEV
TTHSFNCGGEFFYCNTSGLENSTWISNTSVQGSNSTGSNDSLILPCRIKQIINMWQRIGQAMYAPPIQ
GVIRCVSNITGLILTRDGGSINSTTETFRPGGGDMRDNWRSELYKYKVVKIEPLGVAPTRCKRRVVGR
RRRRRAVGIGAVSLGELGAAGSTMGAASMTLTVQARNLLSGIVQQQSNLLRAPEPQQHLLKDTHWGIK
QLQARVLAVEHYLRDQQLLGIWGCSGKLICCTNVPWNSSWSNRNLSEIWDNMTWLQWDKEISNYTQII
YGLLEESQNQQEKNEQDLLALD
Flu HA (NC99) sequence:
(SEQ ID NO: 15; Flu HA (NC99))
CIGYHANNSTDTVDTVLEKNVTVTHSVNLLEDSHNGKLCLLKGIAPLQLGNCSVAGWILGNPECELLI
SKESWSYIVETPNPENGTCEPGYFADYEELREQLSSVSSFERFEIFPKESSWPNHTVTGVSASCSHNG
KSSFYRNLLWLTGKNGLYPNLSKSYVNNKEKEVLVLWGVHHPPNIGNQRALYHTENAYVSVVSSHYSR
RFTPEIAKRPKVRDQEGRINYYWTLLEPGDTIIFEANGNLIAPWYAFALSRGFGSGIITSNAPMDECD
AKCQTPQGAINSSLPFQNVHPVTIGECPKYVRSAKLRMVTGLRNIPQRETRGLFGAIAGFIEGGWTGM
VDGWYGYHHQNEQGSGYAADQKSTQNAINGITNKVNSVIEKMNTQFTAVGKEFNKLERRMENLNKKVD
DGELDIWTYNAELLVLLENERTLDEHDSNVKNLYEKVKSQLKNNAKEIGNGCFEFYHKCNNECMESVK
NGTYDYPKYSEESKINREKIDGGSGYIPEAPRDGQAYVRKDGEWVLLSTFLG

In another embodiment, the disclosure provides polypeptides comprising a sequence selected from the group consisting of (a) CGTKKK-Z1-GGSGGGS-pSer4 (SEQ ID NO:16), pSer4-GGSGGGS-Z1-KKKTGC (SEQ ID NO:17), CGTKKK-maleimide-GGSGGGS-pSer4 (SEQ ID NO:18) and pSer4-GGSGGGS-maleimide-KKKTGC (SEQ ID NO: 19), wherein Z1 is comprises a linkage. In some embodiments, Z1 is selected from the group consisting of maleimide, 5-hydroxy-pyrrolone, a 3-arylpropionitrile, and methylsulfonyl phenyloxadiazole. In these embodiments, the polypeptides can be used, for example to link to an antigen as deemed appropriate by an end-user, to promote coupling the antigen to alum (aluminum hydroxide) via the pSer4 domain with surprisingly strong binding affinity, and providing for improved immunogenicity of the antigen.

In a further embodiment of all of these embodiments, the composition, fusion protein, or polypeptide may be bound to aluminum hydroxide via the pSer4 domain. In this embodiment, the compositions, fusion proteins, or polypeptides are bound to alum, resulting in slowed antigen clearance in vivo, leading to striking increases in germinal center responses and antibody titers as disclosed in the examples that follow.

In another embodiment, the disclosure provides formulations, comprising the composition, fusion protein, or polypeptide bound to aluminum hydroxide via pSer4, and further comprising a non-liposome, non-micelle particle, wherein the particle comprises a lipid, a sterol, a saponin, and an optional additional non-alum adjuvant, wherein the particle is optionally bound to the alum. The particle is optionally bound to the alum. In one embodiment, the alum and the particle are not bound. In another embodiment, the alum and particle are bound. When bound, the alum and particle may be covalently or non-covalently bound. In one embodiment, the particle is covalently bound to the alum via phosphate residues in the particle.

The particle is a non-liposome, non-micelle particle, wherein the particle comprises a lipid, a sterol, a saponin, and an optional additional non-alum adjuvant. Such particles are described, for example, in published US patent application 20200085756, incorporated by reference herein in its entirety. In one embodiment the particle is a porous, cage-like nanoparticle comprising saponin, sterol, lipid, and an optional additional adjuvant. Exemplary saponins, sterols, lipids, additional adjuvants including TLR4 agonists, and antigens are discussed in more detail below. Generally, the nanocage particle is formed by mixing the components together in the presence of a detergent in a suitable ratio such that when the detergent is removed (e.g., by dialysis), the components self-assemble into nanocages. The size of the nanocages is typically dictated by the properties of the components and the self-assembly process. The disclosed compositions and methods typically yield nanocages in the range of about 30 nm and about 60 nm, or about 40 nm to about 50 nm, with an exemplary size being about 40 nm. The nanocages generally assume a distinctive porous morphology that can be structurally distinguished by transmission electronic microscope (TEM) from lipid monolayer (micelle) and lipid bilayer (liposome) particles. The particles are not micelles or liposomes.

The particles include one or more saponins. A suitable saponin is one that can induce or enhance an immune response, Saponins from plants have proven to be very effective as adjuvants. Saponins are triterpene and steroid glycosides widely distributed in the plant kingdom. Structurally, saponins are amphiphilic surfactants, which explains their surfactant properties, ability to form colloidal solutions, hemolytic activity and ability to form mixed micelles with lipids and sterols. The saponins most studied and used as adjuvants are those from Chilean tree Quillaja saponaria, which have cellular and humoral adjuvant activity. Saponins extracts from Quillaja saponaria with adjuvant activity are known and employed in commercial or experimental vaccines formulation.

A particular saponin preparation is called Quil Ax, a saponin preparation isolated from the South American tree Quillaja Saponaria Molina and was first described by Dalsgaard et al. in 1974 (“Saponin adjuvants,” Archiv. für die gesamte Virus forschung, Vol. 44, Springer Verlag, Berlin, p 243-254) to have adjuvant activity. The isolation of pure saponins or better defined mixtures from the Quil A® product having adjuvant activity and lower toxicity than Quil A® have also been described. Purified fragments of Quil A® that retain adjuvant activity without the toxicity associated with Quil A® (EP 0362 278), for example QS7 and QS21 (also known as QA7 and QA21), have been isolated by HPLC. QS-21 is a natural saponin derived from the bark of Quillaja Saponaria Molina, which induces CD8+ cytotoxic T cells (CTLs), Th1 cells and a predominant IgG2a antibody response. QS-21 has been used or is being studied as an adjuvant for various types of vaccines. See also EP 0 362 279 B1 and U.S. Pat. No. 5,057,540.

The isolation and adjuvant activity of other isolated Quil A® saponins, including those called QS-17, and 18 have also been reported, and can also be used in the disclosed nanocages

In other embodiments, the saponin is from Quillaja brasiliensis (A. St.-Hil. et Tul.) Mart., which is native to southern Brazil and Uruguay and has saponins that have proven to be effective as adjuvants with a similar activity against viral antigens as Quil A® (Silveira et al., Vaccine 29 (2011), 9177-9182).

Other useful saponins are derived from the plants Aesculus hippocastanum or Gyophila struthium. Other saponins which have been described in the literature include escin, which has been described in the Merck index (12th ed: entry 3737) as a mixture of saponins occurring in the seed of the horse chestnut tree, Lat: Aesculus hippocastanum. Its isolation by chromatography and purification (Fiedler, Arzneimittel-Forsch. 4, 213 (1953)), and by ion exchange resins (Erbring et al., U.S. Pat. No. 3,238,190) has been described. Fractions of escin have been purified and shown to be biologically active (Yoshikawa M, et al. (Chem Pharm Bull (Tokyo) August 1996; 44(8): 1454-1464)). Sapoalbin from Gypsophila struthium (R. Vochten et al., 1968, J. Pharm. Belg., 42, 213-226) has also been described.

In other embodiments, the saponin is a synthetic saponin. See, e.g., U.S. Published Application No. 2011/0300177 and U.S. Pat. No. 8,283,456, which describe the Triterpene Saponin Synthesis Technology (TriSST) platform, a convergent synthetic approach in which the four domains in QS-21 (branched trisaccharide+triterpene+linear tetrasaccharide+fatty acyl chain) are synthesized separately and then assembled to produce the target molecule. Each of the domains can be modified independently and then combined to produce a virtually infinite number of rationally designed QS-21 analogs. Initially, fully synthetic QS-21(SQS-21) was shown to be safe and immunologically active in a Phase 1 clinical trial, and later over 100 analogues were prepared and tested in a systematic sequential series of studies. See, e.g., Ragupathi, et al., Expert Rev Vaccines, 2011 April; 10(4): 463-470. See also Zu, et al., Journal of Carbohydrate Chemistry, Volume 33, 2014—Issue 6, pages 269-97.

Preferably the saponin component is in a substantially pure form, for example, at least 90% pure, preferably at least 95% pure and most preferably at least 98% pure.

The particles include one or more sterols, Sterols include 0-sitosterol, stigmasterol, ergosterol, ergocalciferol, campesterol, and cholesterol. These sterols are well known in the art, for example cholesterol is disclosed in the Merck Index, 1lth Ed., page 341, as a naturally occurring sterol found in animal fat. In preferred embodiments, the sterol is cholesterol or a derivative thereof e.g., ergosterol or cholesterylhemisuccinate.

The particles include one or more lipids, such as one or more phospholipids. The lipid can be neutral, anionic, or cationic at physiologic pH. Phospholipids include, but are not limited to, diacylglycerides such as phosphatidic acid (phosphatidate) (PA), phosphatidylethanolamine (cephalin) (PE), phosphatidylcholine (lecithin) (PC), phosphatidylserine (PS), and phosphoinositides, e.g., phosphatidylinositol (PI), phosphatidylinositol phosphate (PIP), phosphatidylinositol bisphosphate (PIP2) and phosphatidylinositol trisphosphate (PIP3), as well as phosphoshingolipids such as ceramide phosphorylcholine (Sphingomyelin) (SPH), ceramide phosphorylethanolamine (Sphingomyelin) (Cer-PE), and ceramide phosphoryllipid, and natural and synthetic phospholipid derivatives such as egg PC (Egg lecithin), egg PG, soy PC, hydrogenated soy PC, sphingomyelin, phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), phosphatidylserine (DOPS), and PEG phospholipid (mPEG-phospholipid, polyglycerin-phospholipid, functionalized-phospholipid, terminal activated-phospholipid).

Thus, particles can include any one of more of 1,2-Didecanoyl-sn-glycero-3-phosphocholine (DDPC), 1,2-Dierucoyl-sn-glycero-3-phosphate (Sodium Salt) (DEPA-NA), 1,2-Dierucoyl-sn-glycero-3-phosphocholine (DEPC), 1,2-Dierucoyl-sn-glycero-3-phosphoethanolamine (DEPE) 1,2-Dierucoyl-sn-glycero-3[Phospho-rac-(1-glycerol) (Sodium Salt) (DEPG-NA), 1,2-Dilinoleoyl-sn-glycero-3-phosphocholine (DLOPC), 1,2-Dilauroyl-sn-glycero-3-phosphate (Sodium Salt) (DLPA-NA) 1,2-Dilauroyl-sn-glycero-3-phosphocholine (DLPC) 1,2-Dilauroyl-sn-glycero-3-phosphoethanolamine (DLPE), 1,2-Dilauroyl-sn-glycero-3[Phospho-rac-(1-glycerol) (Sodium Salt) (DLPG-NA), 1,2-Dilauroyl-sn-glycero-3[Phospho-rac-(1-glycerol) (Ammonium Salt) (DLPG-NH4), 1,2-Dilauroyl-sn-glycero-3-phosphoserine (Sodium Salt) (DLPS-NA), 1,2-Dimyristoyl-sn-glycero-3-phosphate (Sodium Salt) (DMPA-NA), 1,2-Dimyristoyl-sn-glycero-3-phosphocholine (DMPC), 1,2-Dimyristoyl-sn-glycero-3-phosphoethanolamine (DMPE), 1,2-Dimyristoyl-sn-glycero-3[Phospho-rac-(1-glycerol) (Sodium Salt) (DMPG-NA) 1,2-Dimyristoyl-sn-glycero-3[Phospho-rac-(1-glycerol) (Ammonium Salt) (DMPG-NH4), 1,2-Dimyristoyl-sn-glycero-3 [Phospho-rac-(1-glycerol) (Sodium/Ammonium Salt) (DMPG-NH4/NA) 1,2-Dimyristoyl-sn-glycero-3-phosphoserine (Sodium Salt) (DMPS-NA), 1,2-Dioleoyl-sn-glycero-3-phosphate (Sodium Salt) (DOPA-NA), 1,2-Dioleoyl-sn-glycero-3-phosphocholine (DOPC, 1,2-Dioleoyl-sn-glycero-3-phosphoethanolamine (DOPE), 1,2-Dioleoyl-sn-glycero-3[Phospho-rac-(1-glycerol) (Sodium Salt) (DOPG-NA), 1,2-Dioleoyl-sn-glycero-3-phosphoserine (Sodium Salt) (DOPS-NA), 1,2-Dipalmitoyl-sn-glycero-3-phosphate (Sodium Salt) (DPPA-NA), 1,2-Dipalmitoyl-sn-glycero-3-phosphocholine (DPPC), 1,2-Dipalmitoyl-sn-glycero-3-phosphoethanolamine (DPPE), 1,2-Dipalmitoyl-sn-glycero-3[Phospho-rac-(1-glycerol) (Sodium Salt) (DPPG-NA), 1,2-Dipalmitoyl-sn-glycero-3 [Phospho-rac-(1-glycerol) (Ammonium Salt) (DPPG-NH4), 1,2-Dipalmitoyl-sn-glycero-3-phosphoserine (Sodium Salt) (DPPS-NA), 1,2-Distearoyl-sn-glycero-3-phosphate (Sodium Salt) (DSPA-NA), 1,2-Distearoyl-sn-glycero-3-phosphocholine (DSPC), 1,2-Distearoyl-sn-glycero-3-phosphoethanolamine (DSPE), 1,2-Distearoyl-sn-glycero-3[Phospho-rac-(I-glycerol) (Sodium Salt) (DSPG-NA), 1,2-Distearoyl-sn-glycero-3[Phospho-rac-(1-glycerol) (Ammonium Salt) (DSPG-NH-14), 1,2-Distearoyl-sn-glycero-3-phosphoserine (Sodium Salt) (DSPS-NA), Egg-PC (EPC), Hydrogenated Egg PC (HEPC), Hydrogenated Soy PC (HSPC), 1-Myristoyl-sn-glycero-3-phosphocholine (LYS OPC MYRISTIC), 1-Palmitoyl-sn-glycero-3-phosphocholine (LYS OPC PALMITIC), 1-Stearoyl-sn-glycero-3-phosphocholine (LYS OPC STEARIC), 1-Myristoyl-2-palmitoyl-sn-glycero 3-phosphocholine (Milk Sphingomyelin MPPC), 1-Myristoyl-2-stearoyl-sn-glycero-3-phosphocholine (MSPC), 1-Palmitoyl-2-myristoyl-sn-glycero-3-phosphocholine (PMPC), 1-Palmitoyl-2-oleoyl-sn-glycero-3-phosphocholine (POPC), 1-Palmitoyl-2-oleoyl-sn-glycero-3-phosphoethanolamine (POPE), 1-Palmitoyl-2-oleoyl-sn-glycero-3[Phospho-rac-(1-glycerol) . . . ](Sodium Salt) (POPG-NA), 1-Palmitoyl-2-stearoyl-sn-glycero-3-phosphocholine (PSPC), 1-Stearoyl-2-myristoyl-sn-glycero-3-phosphocholine (SMPC), 1-Stearoyl-2-oleoyl-sn-glycero-3-phosphocholine (SOPC), and 1-Stearoyl-2-palmitoyl-sn-glycero-3-phosphocholine (SPPC). Any of the lipids can be PEGylated lipids, for example PEG-DSPE. In a specific embodiment, the phospholipid is 2-Dipalmitoyl-snglycero-3-phosphocholine (DPPC).

The formulations and particles may optionally include one or more additional adjuvants (other than alum). In one embodiment, the particle comprises an additional adjuvant. The additional adjuvant typically has physical and biochemical properties compatible with its incorporation into structure of the particle and that do not prevent particle self-assembly. The additional adjuvant also typically increases at least one immune response relative to the same nanocage formulation in the absence of the additional adjuvant. Immune responses include, but are not limited to, an increase in an antigen-specific antibody response (e.g. IgG, IgG2a, IgG1, or a combination thereof), an increase in a response in germinal centers (e.g., increase in the frequency of germinal center B cells, an increase in frequencies and/or activation of T follicular helper (Tfh) cells, an increase in B cell presence or residence in dark zone of germinal center or a combination thereof), an increase in plasmablast frequency, an increase in inflammatory cytokine expression (e.g., TL-6, IFN-γ, IFN-α, IL-1β, TNF-α, CXCL10 (IP-10), or a combination thereof), an increase in drainage of antigen from the injection site, an in increase in antigen accumulation in the lymph nodes, an increase in lymph node permeability, an increase in lymph flow, an increase in antigen-specific B cell antigen uptake in lymph nodes, an increase in humoral responses beyond the proximal lymph node, increased diffusion of antigen into B cell follicles, or a combination thereof, when the nanocages are administered to a subject, preferably in combination with an antigen.

In some embodiments, the additional adjuvant is a TLR agonist. TLR4 is a transmembrane protein member of the toll-like receptor family, which belongs to the pattern recognition receptor (PRR) family. Its activation leads to an intracellular signaling pathway NF-κB and inflammatory cytokine production responsible for activating the innate immune system. Classes of TLR agonists include, but are not limited to, viral proteins, polysaccharides, and a variety of endogenous proteins such as low-density lipoprotein, beta-defensins, and heat shock protein.

Exemplary TLR4 agonist include without limitation derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland).

In another embodiment, the TLR4 agonist is a natural or synthetic lipopolysaccharide (LPS), or a lipid A derivative thereof such as MPLA or 3D-MPLA. Lipopolysaccharides are the major surface molecule of, and occur exclusively in, the external leaflet of the outer membrane of gram-negative bacteria. LPS impede destruction of bacteria by serum complements and phagocytic cells, and are involved in adherence for colonization. LPS are a group of structurally related complex molecules of approximately 10,000 Daltons in size and contain three covalently linked regions: (i) an O-specific polysaccharide chain (O-antigen) at the outer region (ii) a core oligosaccharide central region (iii) lipid A—the innermost region which serves as the hydrophobic anchor, it includes glucosamine disaccharide units which carry long chain fatty acids.

The biological activities of LPS, such as lethal toxicity, pyrogenicity and adjuvanticity, have been shown to be related to the lipid A moiety. In contrast, immunogenicity is associated with the 0-specific polysaccharide component (O-antigen). Both LPS and lipid A have long been known for their strong adjuvant effects, but the high toxicity of these molecules has precluded their use in vaccine formulations. Significant effort has therefore been made towards reducing the toxicity of LPS or lipid A while maintaining their adjuvanticity.

The Salmonella minnesota mutant R595 was isolated in 1966 from a culture of the parent (smooth) strain (Luderitz et al. 1966 Ann. N. Y, Acad. Sci. 133:349-374). The colonies selected were screened for their susceptibility to lysis by a panel of phages, and only those colonies that displayed a narrow range of sensitivity (susceptible to one or two phages only) were selected for further study. This effort led to the isolation of a deep rough mutant strain which is defective in LPS biosynthesis and referred to as S. minnesota R595.

In comparison to other LPS, those produced by the mutant S. minnesota R595 have a relatively simple structure. (i) they contain no O-specific region—a characteristic which is responsible for the shift from the wild type smooth phenotype to the mutant rough phenotype and results in a loss of virulence (ii) the core region is very short—this characteristic increases the strain susceptibility to a variety of chemicals (iii) the lipid A moiety is highly acylated with up to 7 fatty acids.

4′-monophosphoryl lipid A (MPLA), which may be obtained by the acid hydrolysis of LPS extracted from a deep rough mutant strain of gram-negative bacteria, retains the adjuvant properties of LPS while demonstrating a toxicity which is reduced by a factor of more than 1000 (as measured by lethal dose in chick embryo eggs) (Johnson et al. 1987 Rev. Infect. Dis. 9 Suppl:S512-S516). LPS is typically refluxed in mineral acid solutions of moderate strength (e.g. 0.1 M HCl) for a period of approximately 30 minutes. This process results in dephosphorylation at the 1 position, and decarbohydration at the 6′ position, yielding MPLA.

In some embodiments, the TLR4 agonist is MPLA. 3-O-deacylated monophosphoryl lipid A (3D-MPLA), which can be obtained by mild alkaline hydrolysis of MPLA, has a further reduced toxicity while again maintaining adjuvanticity, see U.S. Pat. No. 4,912,094 (Ribi Immunochemicals). Alkaline hydrolysis is typically performed in organic solvent, such as a mixture of chloroform/methanol, by saturation with an aqueous solution of weak base, such as 0.5 M sodium carbonate at pH 10.5. In some embodiments, the TLR4 agonist is 3D-MPLA.

In some embodiments, the MPLA is a fully synthetic MPLA such as Phosphorylated HexaAcyl Disaccharide (PHAD®), the first fully synthetic monophosphoryl Lipid A available for use as an adjuvant in human vaccines, or Monophosphoryl 3-Deacyl Lipid A (Synthetic) (3D-PHAD®). See also U.S. Pat. No. 9,241,988.

As introduced above, the additional adjuvant typically has physical and biochemical properties compatible with its incorporation into the structure of the particle and that do not prevent particle self-assembly and increase an Immune response. Thus, other suitable adjuvants immunostimulators include those that include a lipid tail, or can be modified to contain a lipid tail. Examples of molecules that include a lipid tail, or can be modified to include one, can be, for example, pathogen-associated molecular patterns (PAMPs). PAMPS are recognized by pattern recognition receptors (PRRs). Five families of PRRs have been shown to initiate pro-inflammatory signaling pathways: Toll-like receptors (TLRs), NOD-like receptors (NLRs), RIG-I-like receptors (RLRs), C-type lectin receptors (CLRs) and cytosolic dsDNA sensors (CDSs). Also, some NLRs are involved in the formation of pro-inflammatory complexes called inflammasomes.

Thus, in some embodiments, the additional adjuvant is a TLR ligand, a NOD ligand, an RLR ligand, a CLR ligand, and inflammasome inducer, a STING ligand, or a combination thereof. Such ligands are known in the art can obtained through commercial vendors such as InvivoGen.

As introduced above, the ligands and other adjuvants can be modified (e.g., through chemical conjugation, for example, maleimide thiol reaction, amine N-hydroxysuccinimide ester reaction, click chemistry, etc.) to include a lipid tail to facilitate incorporation of the adjuvant into the nanocage structure during self-assembly, Preferred lipids will include a 160 dipalmitoyl tail such as 1,2-dipalmitoyl-sn-glycero-3-phosphoethanolamine-N-[4-(p-maleimidophenyl)butyramide], these, however, are non-limiting examples. For example, lipids of different lengths are also contemplated. In preferred embodiments, the lipid or lipids is/are unsaturated. Chemically functionalized lipids that that can be used for conjugation are known in the art and commercially available. See, for example, AVANTI® Polar Lipids, Inc. (e.g., “Headgroup Modified Lipids” and “Functionalized Lipids”).

The additional adjuvant can be an immunostimulatory oligonucleotide, preferable a lipidated immunostimulatory oligonucleotide. Exemplary lapidated immunostimulatory oligonucleotides and methods of making them are described in Lin, et al., Nature Letters, 507:519-22 (+11 pages of extended data) (2014)) (lipo-CpG) and U.S. Pat. No. 9,107,904, that contents of which are incorporated by reference herein in their entireties. In some embodiments, the immunostimulatory oligonucleotide portion of the adjuvant can serve as a ligand for PRRs. Therefore, the oligonucleotide can serve as a ligand for a Toll-like family signaling molecule, such as Toll-Like Receptor 9 (TLR9).

For example, unmethylated CpG sites can be detected by TLR9 on plasmacytoid dendritic cells and B cells in humans (Zaida, et al., Infection and Immunity, 76(5):2123-2129, (2008)). Therefore, the sequence of the oligonucleotide can include one or more unmethylated cytosine-guanine (CG or CpG, used interchangeably) dinucleotide motifs. The ‘p’ refers to the phosphodiester backbone of DNA, as discussed in more detail below, some oligonucleotides including CG can have a modified backbone, for example a phosphorothioate (PS) backbone.

In some embodiments, an immunostimulatory oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CC dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, A M Advanced drug delivery reviews 61(3): 195-204 (2009), incorporated herein by reference). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, G L, PNAS USA 94(20): 10833-7 (1997); Dalpke, A H, Immunology 106(1): 102-12 (2002); Hartmann, C, J of Immun. 164(3):1617-2 (2000), each of which is incorporated herein by reference).

Other PRR Toll-like receptors include TLR3, and TLR7 which may recognize double-stranded RNA, single-stranded and short double-stranded RNAs, respectively, and retinoic acid-inducible gene I (RIG-I)-like receptors, namely RIG-I and melanoma differentiation-associated gene 5 (MDAS), which are best known as RNA-sensing receptors in the cytosol. Therefore, in some embodiments, the oligonucleotide contains a functional ligand for TLR3, TLR7, or RIG-I-like receptors, or combinations thereof.

Examples of immunostimulatory oligonucleotides, and methods of making them are known in the art, see for example, Bodera, P. Recent Pat Inflamm Allergy Drug Discov. 5(1):87-93 (2011), incorporated herein by reference.

In some embodiments, the oligonucleotide includes two or more immunostimulatory sequences.

Microbial cell-wall components such as Pam2CSK4, Pam3CSK4, and flagellin activate TLR2 and TLR5 receptors respectively and can also be used.

Any suitable ratios of the various particle components may be used. In one embodiment, comprising a lipid: additional adjuvant:sterol:saponin molar ratio of 2.5:1:10:10, or a variation thereof wherein the molar ratio of lipid, additional adjuvant, sterol, saponin or any combination thereof is increased or decreased by any value between about 0 and about 3. In a specific embodiment, the lipid is DPPC, the additional adjuvant is a natural or synthetic MPLA, the sterol is cholesterol, and the saponin is Quil A® in a molar ratio of 2.5:1:10:10. In another embodiment

the Quil-A:chol:DPPC:MPLA are in a mass ratio of 10:2:1:1. See US20200085756 for exemplary methods for modifying the molar ratio or mass ratio of the particle components.

In a further embodiment, the disclosure provides pharmaceutical compositions comprising the composition, fusion protein, polypeptide, or formulation of any embodiment of the disclosure, and a pharmaceutically acceptable carrier. Suitable acids which are capable of forming such salts include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming such salts include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanol-amines (e.g., ethanolamine, diethanolamine and the like).

In some embodiments, the pharmaceutical composition can contain components for modifying, maintaining or preserving, for example, the pH, osmolality, viscosity, clarity, color, isotonicity, odor, sterility, stability, rate of dissolution or release, adsorption or penetration of the composition. In some embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences. 18th Edition, A. R. Gennaro, ed., Mack Publishing Company (1995). In certain embodiments, the formulation comprises PBS; 20 mM NaOAC, pH 5.2, 50 mM NaCl; and/or 10 mM NAOAC, pH 5.2, 9% Sucrose, in some embodiments, the optimal pharmaceutical composition will be determined by one skilled in the art depending upon, for example, the intended route of administration, delivery format and desired dosage. See, for example, Remington's Pharmaceutical Sciences, supra. In some embodiments, such compositions may influence the physical state, stability, rate of in vivo release and rate of in vivo clearance of the immunogenic composition.

In some embodiments, the primary vehicle or carrier in a pharmaceutical composition can be either aqueous or non-aqueous in nature. For example, in some embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In certain embodiments, neutral buffered saline or saline mixed with serum albumin are further exemplary vehicles. In some embodiments, pharmaceutical compositions comprise Tris buffer of about pH 7.0-8.5, or acetate buffer of about pH 4.0-5.5, which can further include sorbitol or a suitable substitute therefore. In some embodiments, an immunogenic composition can be prepared for storage by mixing the selected composition having the desired degree of purity with optional formulation agents (Remington's Pharmaceutical Sciences. supra) in the form of a lyophilized cake or an aqueous solution. Further, in some embodiments, an immunogenic composition can be formulated as a lyophilizate using appropriate excipients such as sucrose.

The pharmaceutical compositions of the invention may be made up in any suitable formulation, preferably in formulations suitable for administration by parenteral delivery such as subcutaneous of intra-venous injection, inhalation, or oral delivery. Such pharmaceutical compositions can be used, for example, in the therapeutic methods disclosed herein.

The pharmaceutical compositions may contain any other components as deemed appropriate for a given use. In another embodiment, the disclosure provides vaccines comprising the composition or fusion protein of any embodiment of the disclosure in which an antigen is present. The compositions and vaccines may be used, for example in the methods of the disclosure.

In another aspect, the disclosure provides methods for generating an immune response against an antigen, comprising administering to a subject an amount effective to generate an immune response in the subject of the composition, fusion protein, polypeptide, formulation, pharmaceutical composition, or vaccine of any embodiment herein.

The “immune response” refers to responses that induce, increase, or perpetuate the activation or efficiency of innate or adaptive immunity. The immune response includes, but is not limited to, the production of antibodies and/or cytokines and/or the activation of cytotoxic T cells, antigen presenting cells, helper T cells, dendritic cells and/or other cellular responses.

In a further embodiment, the disclosure provides methods of treating a subject in need thereof comprising administering to the subject the composition, fusion protein, polypeptide, formulation, pharmaceutical composition, or vaccine of any embodiment herein in an effective amount to induce an immune response against the antigen.

In some embodiments, the immunogenic compositions are administered as part of prophylactic vaccines or immunogenic compositions which confer resistance in a subject to subsequent exposure to infectious agents, or as pail of therapeutic vaccines, which can be used to initiate or enhance a subject's immune response to a pre-existing antigen, such as a viral antigen in a subject infected with a with an infectious agent or neoplasm. The desired outcome of a prophylactic or therapeutic immune response may vary according to the disease or condition to be treated, car according to principles well known in art. For example, an immune response against an infectious agent may completely prevent colonization and replication of an infectious agent, affecting “sterile immunity” and the absence of any disease symptoms. However, a vaccine against infections agents may be considered effective if it reduces the number, severity or duration of symptoms; if it reduces the number of individuals in a population with symptoms; or reduces the transmission of an infectious agent. Similarly, immune responses against cancer, allergens or infectious agents may completely treat a disease, may alleviate symptoms, or may be one facet in an overall therapeutic intervention against a disease.

Methods for analyzing an antibody response in a subject are known to those of skill in the art. For example, in some embodiments an increase in an immune response is measured by ELISA assays to determine antigen-specific antibody titers. In some embodiments, the methods increasing broadly neutralizing antibodies in a subject. Methods for measuring neutralizing antibodies are known to those of ordinary skill in the art. In some embodiments, elicitation of neutralizing antibodies is measured in a neutralization assay. Methods for identifying and measuring neutralizing antibodies are known to those of skill in the art. Neutralizing antibodies are an indicator of the protective efficacy of a vaccine, but direct protection from a sub-lethal or lethal challenge of virus unequivocally demonstrates the efficacy of the vaccine.

In some embodiments, the administration of the composition, fusion protein, polypeptide, formulation, pharmaceutical composition, or vaccine induces an improved B-memory cell response in immunized subjects. An improved B-memory cell response is intended to mean an increased frequency of peripheral blood B lymphocytes capable of differentiation into antibody-secreting plasma cells upon antigen encounter as measured by stimulation of in vitro differentiation. In some embodiments, the methods increase the number of antibody secreting B cells. In some embodiments, the antibody secreting B cells are bone marrow plasma cells, or germinal center B cells. In some embodiments, methods for measuring the number of antibody secreting B cells, includes, but are not limited to, an antigen-specific ELISPOT assay and flow cytometric studies of plasma cells, or germinal center B cells collected at various time points post-immunization.

In some embodiments, the composition, fusion protein, polypeptide, formulation, pharmaceutical composition, or vaccine described herein (when the antigen is a tumor antigen). is useful for treating a disorder associated with abnormal apoptosis or a differentiative process (e.g., cellular proliferative disorders (e.g., hyperproliferative disorders) or cellular differentiative disorders, such as cancer).

In other embodiments, the composition, fusion protein, polypeptide, formulation, pharmaceutical composition, or vaccine disclosed herein is useful for treating or limiting development of acute or chronic infectious diseases. Thus, in some embodiments an immunogenic composition or vaccine is administered for the treatment of local or systemic viral infections, including, but not limited to, immunodeficiency (e.g., HIV) and influenza (e.g., human influenza virus A), viral infections.

In some embodiments, the disclosure provides methods of reducing or limiting a viral infection in a subject in need thereof, comprising administering to the subject a composition, fusion protein, polypeptide, formulation, pharmaceutical composition, or vaccine described herein. In some embodiments, the disclosure provides methods for inducing an anti-viral response in a subject with cancer, comprising administering to the subject an immunogenic composition or vaccine described herein.

The “subject” may be any human or non-human animal.

### Example 1

In the ongoing effort to develop a vaccine against HIV, vaccine approaches that promote strong germinal center (GC) responses may be critical to enable the selection and affinity maturation of rare B cell clones capable of evolving to produce broadly neutralizing antibodies. We previously demonstrated an approach for enhancing GC responses and overall humoral immunity elicited by alum-adjuvanted protein immunization, via the use of phosphoserine (pSer) peptide-tagged immunogens that stably anchor to alum particles via ligand exchange with the alum particle surface. Here, using a clinically relevant stabilized HIV Env trimer termed MD39, we systematically evaluated the impact of several parameters relevant to pSer tag composition and trimer immunogen design to improve this approach including phosphate valency, amino acid sequence of the trimer C-terminus used for pSer tag conjugation, and structure of the pSer tag. We also tested the impact of co-administering a potent saponin/monophosphoryl lipid A (MPLA) nanoparticle co-adjuvant with alum-bound trimers, We identified MD39 trimer sequences bearing; a positively-charged C-terminal amino acid sequence, which when conjugated to a pSer tag with four phosphates and a polypeptide spacer, bound very tightly to alum particles while retaining a native Env-like antigenicity profile. This improved pSer-trimer design elicited robust antigen-specific GC B cell and serum IgG responses in mice, a significant improvement over previous designs and suitable for clinical use.

The human immunodeficiency virus/acquired immunodeficiency syndrome (HIV/AIDS) epidemic began more than 40 years ago. Despite the increasing availability of antiretroviral drugs, HIV remains a leading cause of death globally, with over 37 million people currently infected with HIV worldwide. As a result, there is a significant unmet need for the development of an effective prophylactic HIV vaccine as a low-cost, facile solution to prevent infection. Due to the diversity and high mutational capacity of the virus, the induction of broadly neutralizing antibodies (bnAbs) in the context of vaccination has remained a challenge.

We recently investigated a strategy to stably bind antigen to aluminum hydroxide (alum) particles:

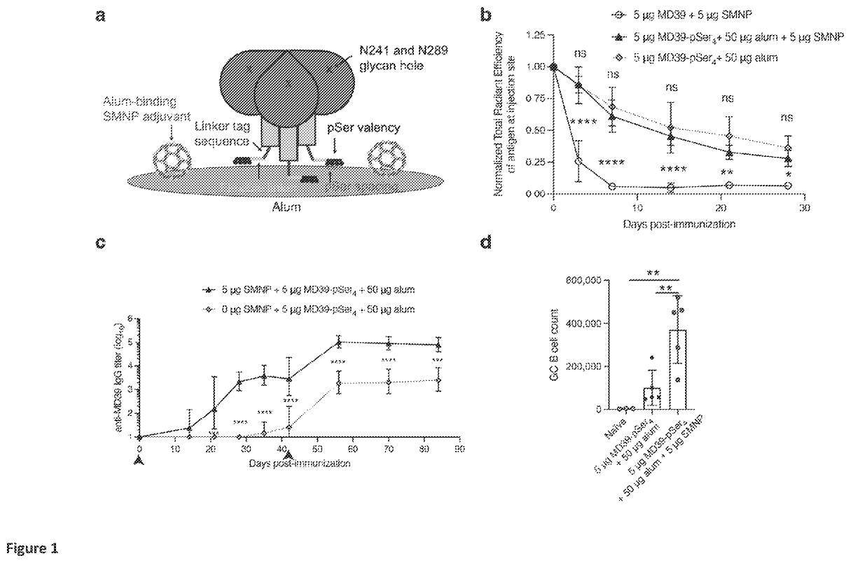

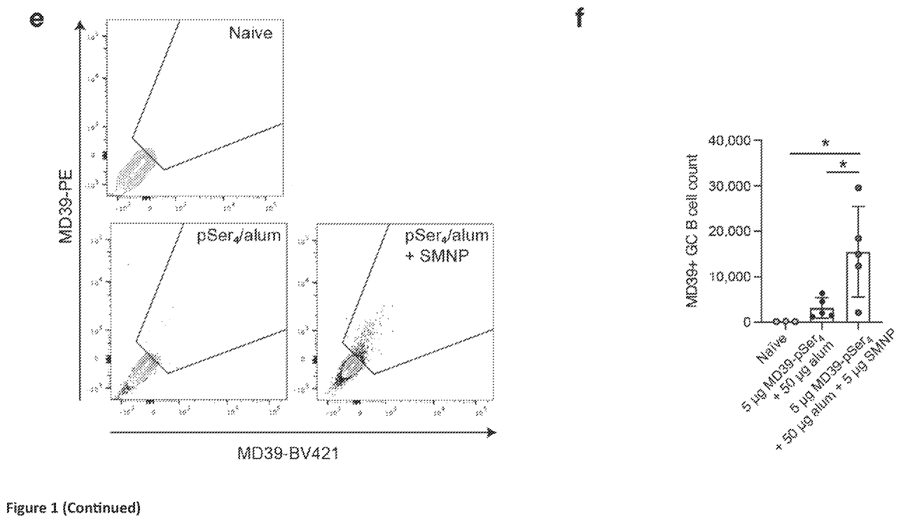

We sought to position the pSer-antigen/alum technology for clinical translation, exemplified with a hyperstabilized HIV Env SOSIP trimer termed MD39, which is a current clinical candidate. Our prior proof-of-concept studies were performed using Env trimers modified with C-terminal His-tags capped by a cysteine for chemical attachment of pSer tags at the base of the trimer. However, His-tags are immunogenic and undesirable for clinical vaccine products. We also hypothesized that the design of the pSer tag itself might be improved in terms of composition, phosphate valency, or phosphate spacing to mediate maximal alum binding. Thus, here we explored the design of the immunogen and pSer tag to maximize on-target, vaccine-relevant responses and minimize undesired responses to vaccine-irrelevant epitopes (FIG. 1a). We first investigated the impact of phosphate valency on the in vitro alum binding of MD39 as well as in vivo humoral immune responses, and tested the impact of combining alum delivery with a promising saponin nanoparticle-based adjuvant, SMNP (saponin/phospholipid nanoparticle adjuvant). We subsequently screened linkers expressed on the immunogen to replace the polyhistidine purification sequence used in our preclinical immunization studies, analyzing humoral responses to a set of downselected constructs which behaved favorably in vitro. The immunogen construct was further polished by filling a glycan hole.²¹ Finally, we assessed the impact of phosphate spacing in the pSer tag and composition of the linker. Together, these studies identified an improved MD39-pSer for clinical use.

### Results

Synergistic Immune Priming by Combining MD39-pSer/Alum with an Alum-Binding Saponin/TLR-4 Agonist Nanoparticle Adjuvant

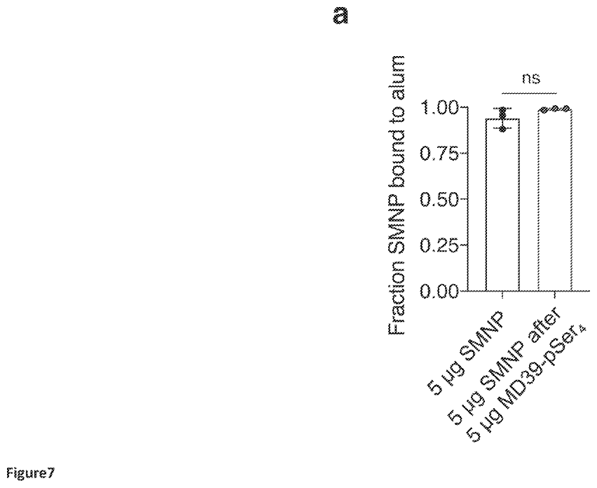

In our initial proof-of-concept studies, we established a strategy to anchor stabilized HIV Env trimers to alum particles by chemically conjugating pSer peptide tags at the base of the trimer (FIG. 1a). pSer-tagged trimers are prepared by reaction of a short maleimide-functionalized phosphoserine-containing peptide to a free cysteine introduced at the C-terminus of the MD39 gp140 protomers (Table 1). In order to further boost responses to alum-anchored trimers, we investigated the effect of adding a nanoparticle adjuvant which is also capable of undergoing ligand exchange with alum. SMNP (Saponin/MPLA nanoparticle) is an ISCOMs-like nanoparticle ~40 nm in diameter comprised of saponin and the Toll like receptor (TLR)-4 agonist monophosphoryl lipid A (MPLA) self-assembled with lipids and cholesterol,²⁴ which we previously showed could bind stably to alum in concert with pSer-tagged immunogens via ligand exchange between the phospholipid headgroups of the particle and alum.¹⁹ We observed near-complete retention of SMNP on alum after 24-hour incubation in phosphate buffer containing 10% mouse serum, regardless of the initial loading of MD39-pSer₄(MD39 trimer tagged with a peptide tag containing 4 phosphoserine residues, FIG. 7a), consistent with our previous studies of pSer-tagged SARS-CoV-2 S receptor binding domain immunogens.¹⁹ We next immunized mice with fluorescently labeled MD39-pSer₄ bound to alum and tracked the clearance of antigen from the injection site using whole-animal fluorescence imaging. In these experiments, the addition of SMNP to the alum/MD39-pSer₄ formulation did not substantially alter antigen drainage kinetics; both groups combining alum with the pSer-conjugated trimer showed much slower clearance of antigen from the injection site than the soluble MD39 trimer/SMNP comparator group (FIG. 1b). To assess the impact of SMNP as a co-adjuvant on humoral responses, we immunized mice with MD39-pSer₄/alum combined with SMNP and boosted the animals at 6 weeks, tracking serum anti-MD39 IgG antibody responses longitudinally by ELISA. Addition of SMNP markedly improved antibody responses, leading to seroconversion of all animals post-prime (FIG. 1c). Further, SMNP significantly enhanced GC B cell responses (FIG. 1d-f), inducing striking increases in MD39-specific. GC B cell responses primed by MD39-pSer₄/alum immunizations. Thus, SMNP enhances multiple facets of humoral immune responses following MD39-pSer₄/alum vaccination.

TABLE 1
MD39_his with C-terminal cysteine

ETGAENLWVTVYYGVPVWKDAETTLFCASDAKAYETEKHNVWATHACVPTDPNPQEIHLEN
VTEEFNMWKNNMVEQMHEDIISLWDQSLKPCVKLTPLCVTLQCTNVTNNITDDMRGELKNC
SFNMTTELRDKKQKVYSLFYRLDVVQINENQGNRSNNSNKEYRLINCNTSAITQACPKVSF
EPIPIHYCAPAGFAILKCKDKKFNGTGPCPSVSTVQCTHGIKPVVSTQLLLNGSLAEEEVI
IRSENITNNAKNILVQLNTPVQINCTRPNNNTVKSIRIGPGQAFYYTGDIIGDIRQAHCNV
SKATWNETLGKVVKQLRKHFGNNTIIRFAQSSGGDLEVTTHSFNCGGEFFYCNTSGLFNST
WISNTSVQGSNSTGSNDSITLPCRIKQIINMWQRIGQAMYAPPIQGVIRCVSNITGLILTR
DGGSTNSTTETFRPGGGDMRDNWRSELYKYKVVKIEPLGVAPTRCKRRVVGRRRRRRAVGI
GAVSLGFLGAAGSTMGAASMTLTVQARNLLSGIVQQQSNLLRAPEPQQHLLKDTHWGIKQL
QARVLAVEHYLRDQQLLGIWGCSGKLICCTNVPWNSSWSNRNLSEIWDNMTWLQWDKEISN
YTQIIYGLLEESQNQQEKNEQDLLALDGTKHHHHHHC (SEQ ID NO: 21)

Role of pSer Valency in Modulating Alum Binding, Antigen Clearance Rates In Vivo, and Vaccine Immunogenicity

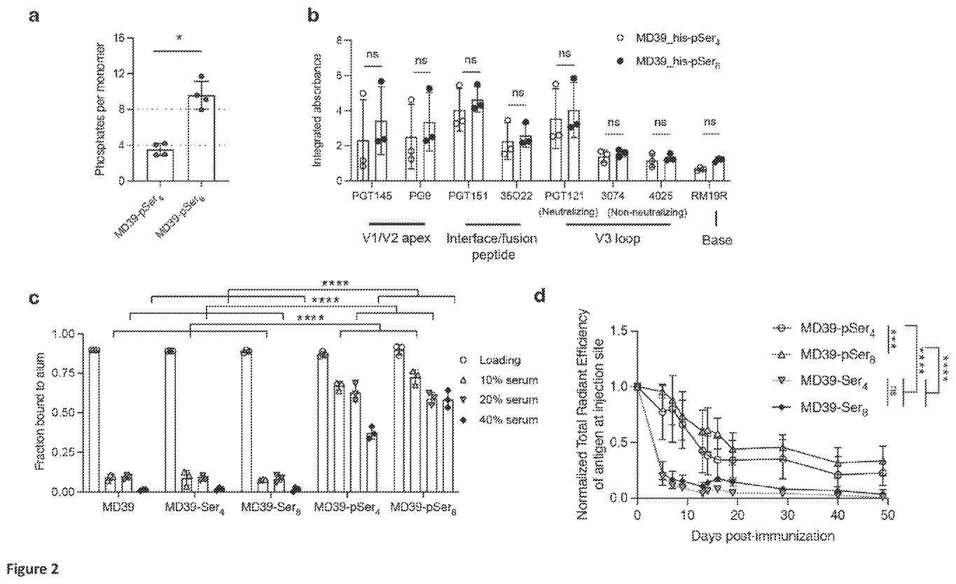

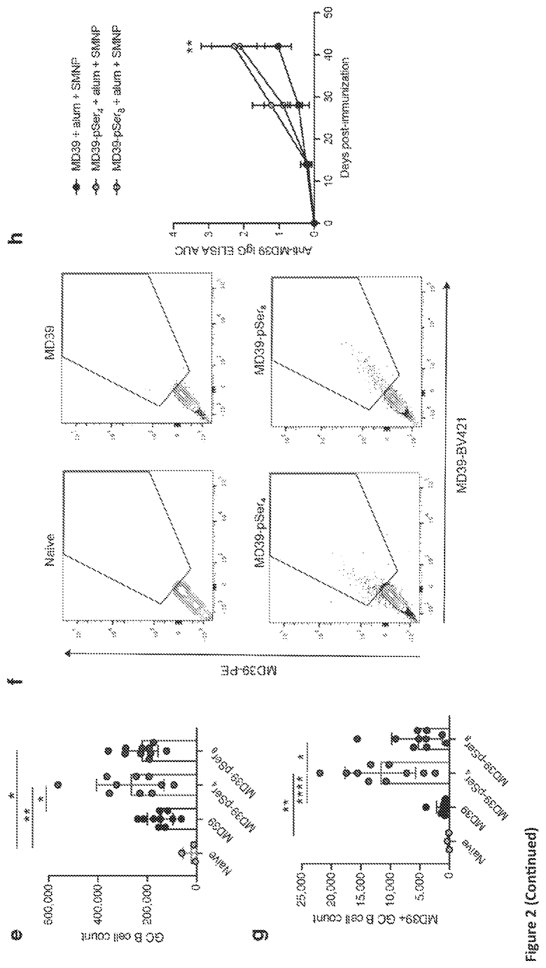

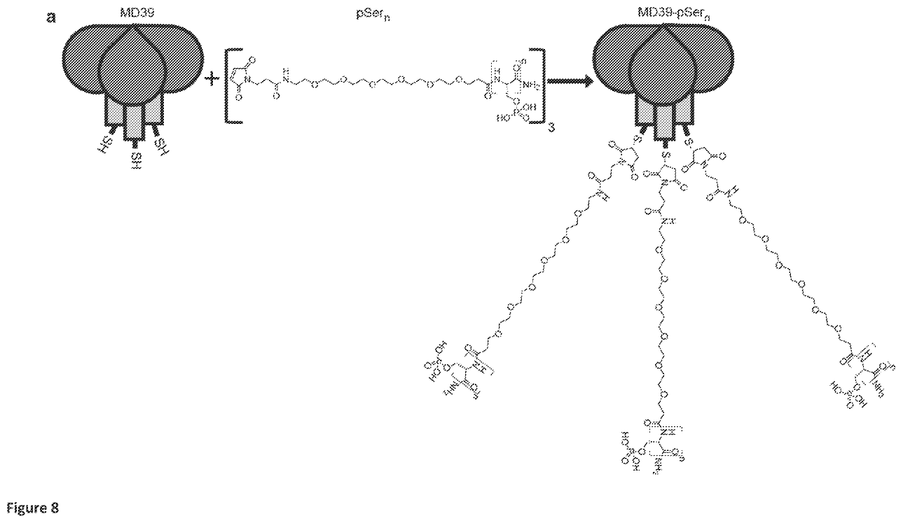

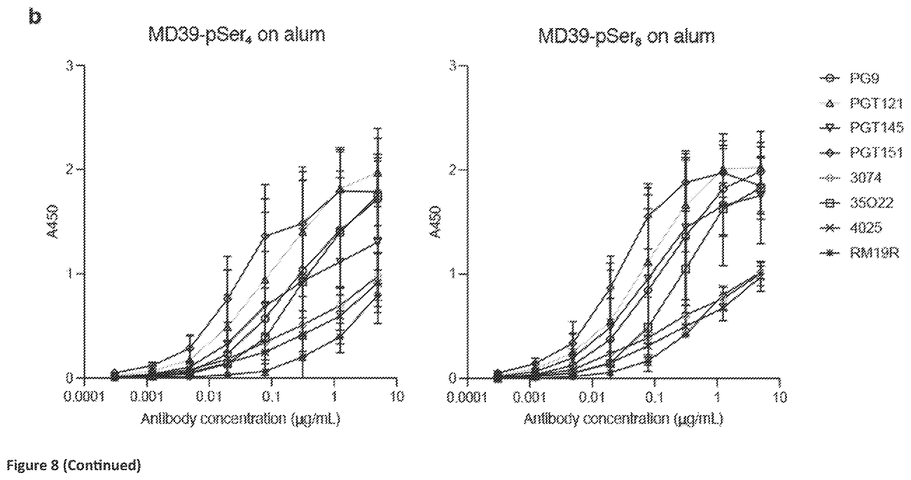

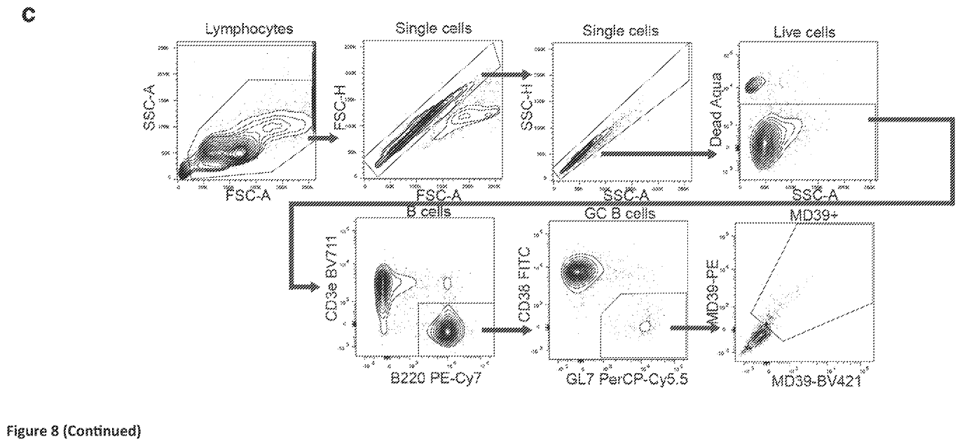

Since the interactions between pSer-antigens and alum are mediated by the phosphate groups on pSer linkers, we next investigated the impact of pSer valency (number of phosphates in the affinity tag) on the stability of trimer binding to alum (FIG. 8a). We previously found that tags containing four phosphoserines (pSer₄) were effective at stably binding monomeric and trimeric immunogens to alum.^{18,19} However, we hypothesized there could be a benefit to further increasing the valency of the pSer tag. To investigate this, peptide tags containing 4 or 8 pSer residues were synthesized, and the impact of pSer valency on alum binding in vitro was investigated. Measurement of the mean number of phosphates per protomer using a malachite green assay revealed 3.5 and 9.6 phosphates per MD39 protomer for MD39-pSer₄ and MD39-pSer₈, respectively, near the expected ~4 and ~8 phosphates per promoter (FIG. 2a). To confirm that phosphoserine-tagged timers retained structural integrin upon binding to alum, we used a modified sandwich ELISA approach to probe the antigenicity profiles of the constructs: pSer-conjugated MD39 was captured on alum-coated plates, and the immobilized immunogen was probed for binding to serial dilutions of a panel of neutralizing and non-neutralizing monoclonal antibodies, This analysis revealed comparable antigenicity profiles for MD39-pSer₄ and -pSer₈ on alum for V1/V2 apex, interface/fusion peptide, and neutralizing V3 loop epitopes (FIG. 2b, 8b). Importantly, both pSer-conjugated trimers exhibited low binding to antibodies targeting non-neutralizing V3 loop epitopes and the trimer base. The latter is expected if anchoring the trimers to alum sterically obscures the base of the immunogen, which is important since the base of soluble HIV Env trimers is known to be a highly immunogenic surface that can be

immunodominantly targeted by the antibody response and irrelevant for protection.²⁵ Next we assessed the stability of alum binding for the pSer-tagged immunogens. MD39 trimers were incubated with alum for 30 minutes in tris-buffered saline to allow adsorption to the alum particles, followed by incubation for 24 hours in increasing concentrations of mouse serum at 37° C. to test binding stability. Unmodified MD39 showed poor retention on alum, with the majority of antigen desorbed following incubation with only 10% mouse serum (FIG. 2c. In comparison, the pSer-modified antigens exhibited much greater retention on alum in the presence of serum, and the higher phosphate valency MD39-pSer₈ showed greater retention on alum than MD39-pSer₄.

Next, we investigated whether these differences in alum binding observed in vitro translated to differential antigen clearance kinetics in vivo. Mice were immunized subcutaneously (s.c.) at the tail base with fluorophore-labeled MD39-pSer₄ or MD39-pSer₈ mixed with alum, or control serine-tagged MD39-Ser₄, or MD39-Ser₄ and alum, and the kinetics of antigen clearance from the injection site were tracked longitudinally by whole animal fluorescence imaging. For the control Ser₄- and Ser₈-conjugated trimers that are incapable of ligand exchange-mediated binding to alum, we observed rapid antigen clearance from the immunization site, with only ~20% of the antigen remaining by 5 days post injection (FIG. 2d), By contrast, MD39-pSer₄ and MD39-pSer₈ both exhibited significantly increased persistence at the injection site, with a steady decay over ~3 weeks followed by slower clearance at later times; MD39-pSer₈/alum cleared slightly slower than MD39-pSer₄/alum (p<0.001, FIG. 2d).

To assess whether these differences in antigen drainage kinetics impact the resulting humoral immune response, we immunized mice with MD39, MD39-pSer₄, or MD39-pSer₈ mixed with alum, and carried out flow cytometry analysis of germinal center (GC) B cell responses in draining inguinal lymph nodes at 14 days post-immunization (FIG. 8c). Although both unmodified and pSer-tagged MD39 elicited substantial total CC B cell responses (FIG. 2e), trimer-binding CC B cells were barely detectable at this time point for immunization with non-tagged trimer and alum (FIG. 2f-g). Notably, the absolute magnitude of trimer-specific GC B cells was significantly greater for MD39-pSer₄/alum immunization and trended toward more than double the response elicited by MD39-pSer₈/alum (FIG. 2f-g). Next, we immunized mice with MD39, MD39-pSer₄, or MD39-pSer₈ mixed with alum plus SMNP to maximize the humoral response, tracking the serum anti-MD39 IgG antibody response (FIG. 2h). The serum IgG titers elicited by MD39-pSer₈/alum immunization were comparable to MD39-pSer₄/alum. Thus, we focused on a 4-valent phosphoserine tag for subsequent studies.

Modifying the C-Terminal Sequence of MD39 Trimers for pSer Tag Conjugation

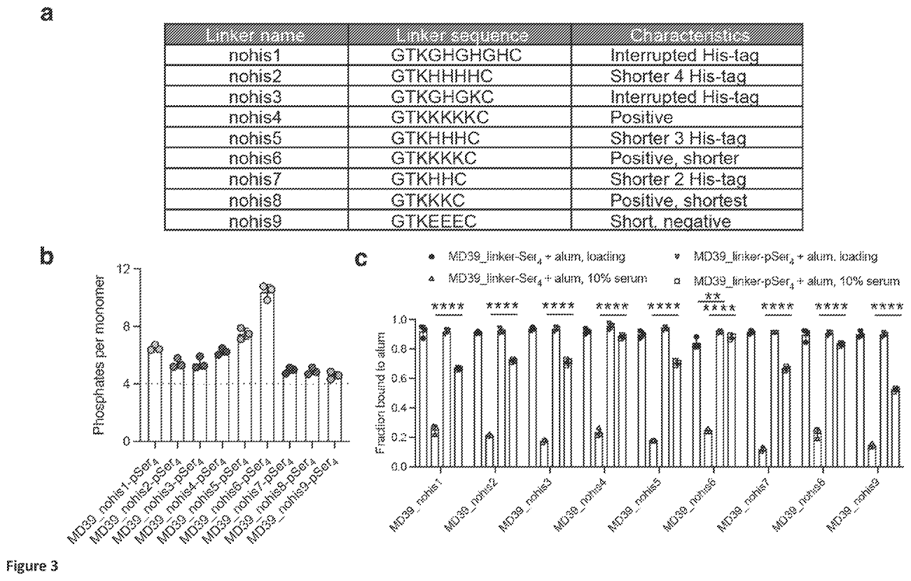

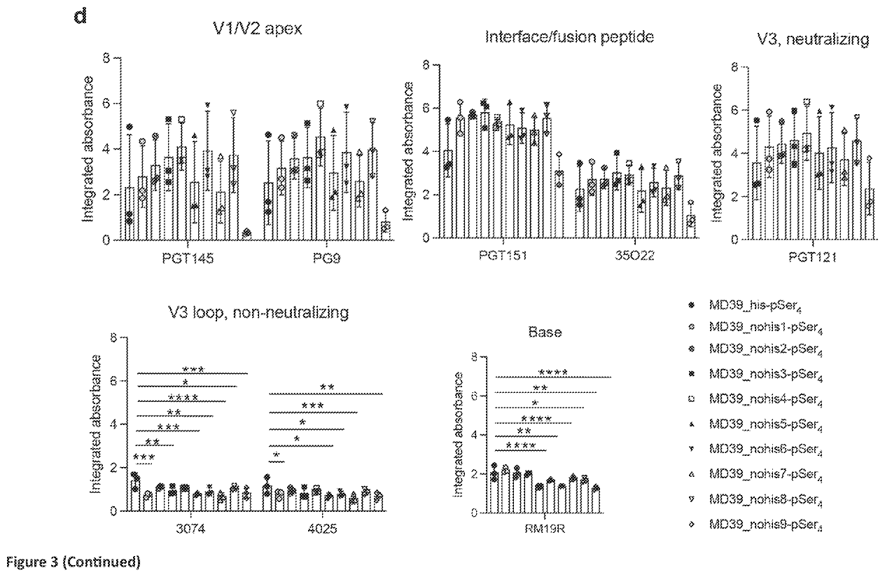

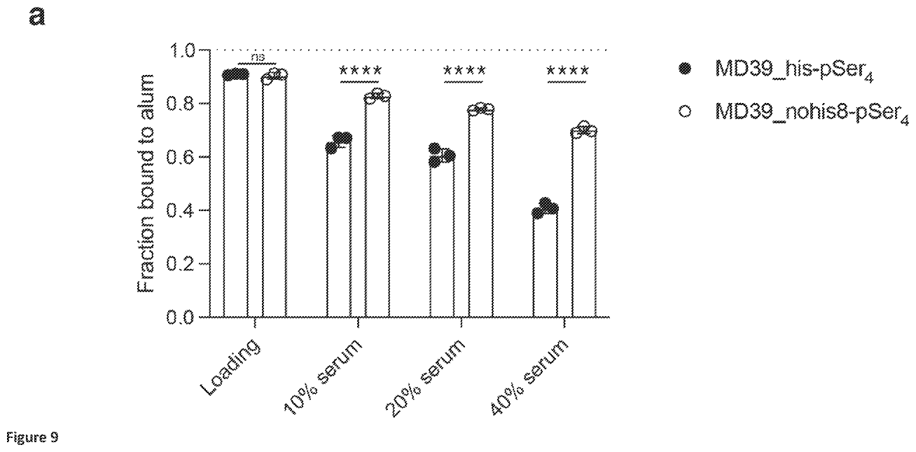

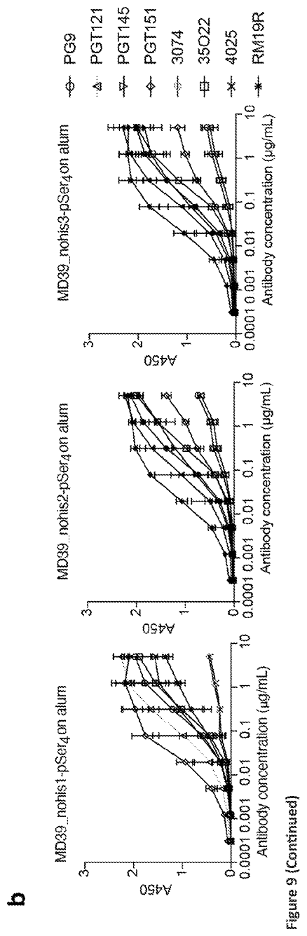

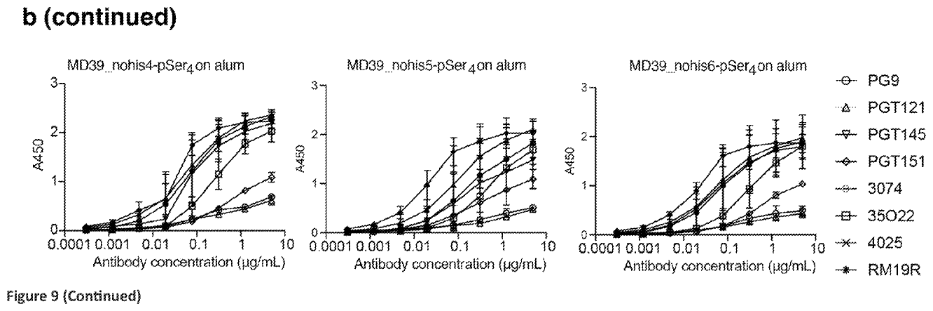

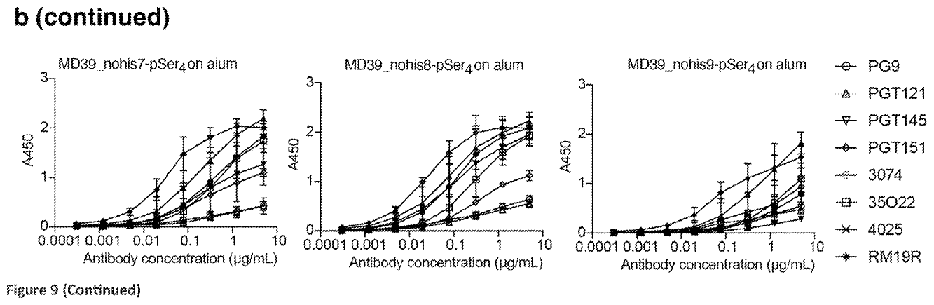

Our preclinical studies to this point employed trimer proteins bearing a C-terminal His-tag sequence just before the cysteine residue used for pSer peptide coupling. While incorporation of an N- or C-terminal polyhistidine tag is commonly used to facilitate purification of recombinant proteins using immobilized metal ion affinity chromatography for preclinical studies, His-tags can be immunogenic.²³ This response has the potential to be immunodominant, such that B cells which recognize the polyhistidine linker could outcompete those which recognize more relevant antigen-specific epitopes. To replace the polyhistidine linker, we synthesized MD39 constructs incorporating a panel of 9 alternate cysteine-terminated C-terminal spacers varying in length and overall charge, and screened these proteins for pSer conjugation, alum binding, and antigenicity profiles (FIG. 3a). Each of these immunogens were conjugated to pSer₄ tags and the degree of labeling was assessed by a malachite green assay. Several of the constructs showed over-labeling with significantly more than the target ~4 mean phosphates per protomer, including MD39_nohis5 and MD39_nohis6, suggesting that the maleimide-pSer tag was coupling to additional residues (e.g., lysine primary amines) and not just the terminal free cysteine (FIG. 3b).^{26,27} To assess the impact of the immunogen linker on alum binding behavior, pSer₄-conjugated or unmodified MD39 constructs were mixed with alum, and the fraction of protein bound to alum was assessed before and after incubation for 24 hours in 10% mouse serum at 37° C. Several of the trimers bearing “nohis” linkers exhibited very stable binding to alum, with up to ~90% of the pSer-modified antigen remaining bound after serum incubation (e.g., MD39 nohis4, MD39_nohis6, MD39 nohis8, FIG. 3c). By contrast MD39_nohis9-pSer₄ demonstrated weaker retention on alum compared to other constructs (FIG. 3c). Excluding the over-labeled MD39_nohis6-pSer₄, MD39_nohis4-pSer₄ and MD39_nohis8-pSer₄ exhibited the best retention on alum (FIG. 3c) and showed more stable binding to alum in the presence of serum than our original MD39_his-pSer₄ construct (FIG. 9a). To assess whether the alum-bound MD39 nohis pSer-conjugates were structurally intact when bound to alum, we evaluated the antigenicity profiles of the alum-bound constructs. While most constructs retained major epitopes indicative of structurally intact antigen. construct MD39_nohis9-pSer in particular exhibited substantially lower binding of antibodies targeting the V1/V2 apex, interface/fusion peptide, and neutralizing V3 epitopes, suggesting partial unfolding of this trimer construct upon pSer-mediated alum binding (FIG. 3d, 9b). Interestingly, the non-histag-containing MD39 constructs 4-9 showed lower binding of the trimer base-specific mAb RM19R, suggesting that they achieved more effective blocking of the trimer base when immobilized on alum. Considering the alum binding data, constructs MD39 nohis4 and MD39_nohis8 appeared most promising given strong V1/V2 apex, interface/fusion peptide, and neutralizing V3 epitope binding while shielding non-neutralizing epitopes. As a result, we sought to move forward with in vivo investigation of humoral responses to these two constructs.

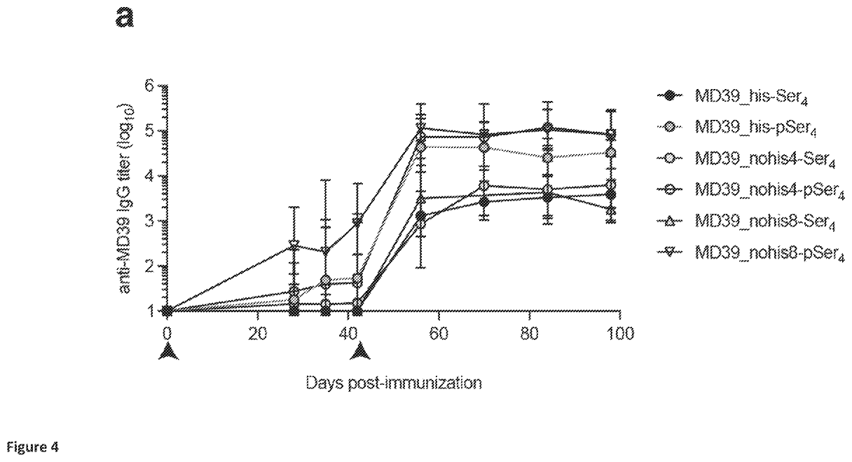

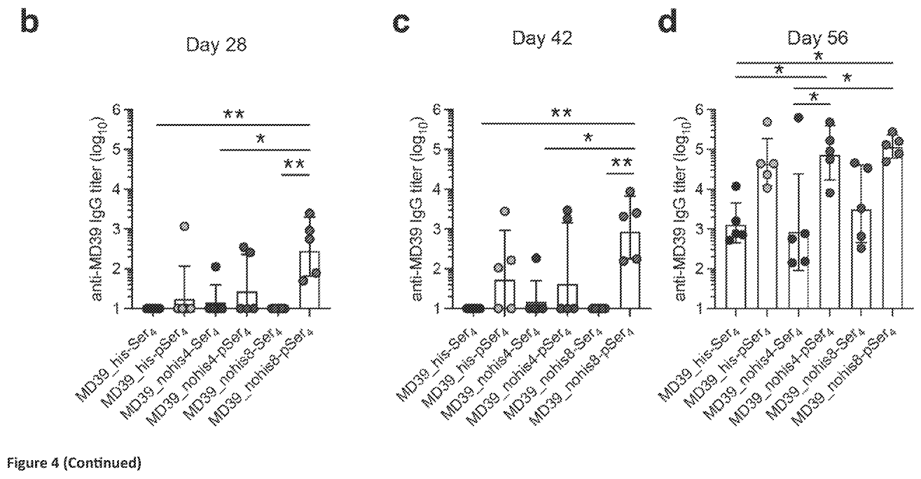

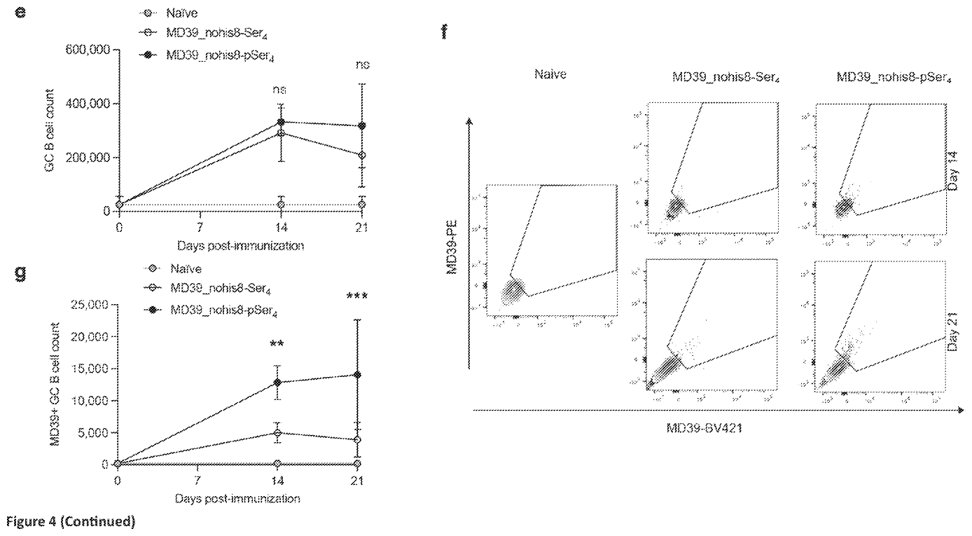

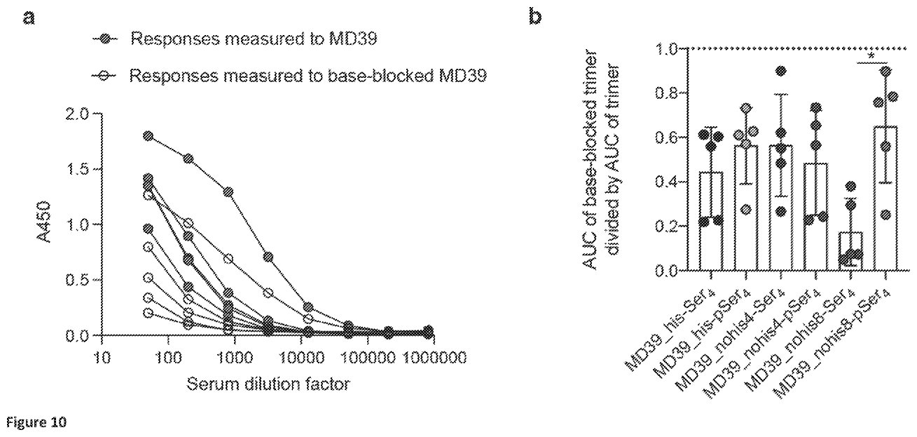

We first assessed the immunogenicity of these modified trimers in BALB/c mice compared to MD39 containing a polyhistidine linker. Mice were immunized with Ser₄- or pSer₄-conjugated trimers and alum and then boosted 6 weeks later with the same constructs combined with alum and SMNP (FIG. 4a). Post-prime, MD39_nohis8-pSer₄ elicited significantly stronger responses compared to all other groups, with seroconversion observed in all animals immunized with MD39_nohis8-pSer₄vs. only 3 of 5 animals receiving his-tagged MD39 and 2 of 5 animals receiving MD39_nohis4-pSer₄ (FIG. 4b-c). Two weeks post-boost, there was no significant difference in anti-MD39 serum IgG titers between the three pSer₄-conjugated MD39 constructs, though MD39_nohis8-pSer₄ elicited the most consistent high titers (FIG. 4d). Further, we assessed the proportion of the antibody response directed against the trimer base via ELISA assays using plate-bound trimer in the presence or absence of a base-blocking monoclonal antibody (FIG. 10a), and found that MD39_nohis8-pSer₄ elicited the lowest base response (FIG. 10b). We next assessed MD39-specific germinal center (GC) responses at days 14 and 21 post immunization in mice immunized with Ser₄- or pSer₄-conjugated MD39 nohis8 combined with alum and SMNP. Flow cytometry analysis of draining inguinal lymph nodes revealed strong total GC responses in mice immunized with either control Ser₄-trimers or alum-binding pSer₄-conjugated trimers, but the pSer-tagged trimer elicited a significantly stronger antigen-specific CC B cell response that was sustained from day 14 to day 21 (FIG. 4e-g).

Modifying pSer Tag Design

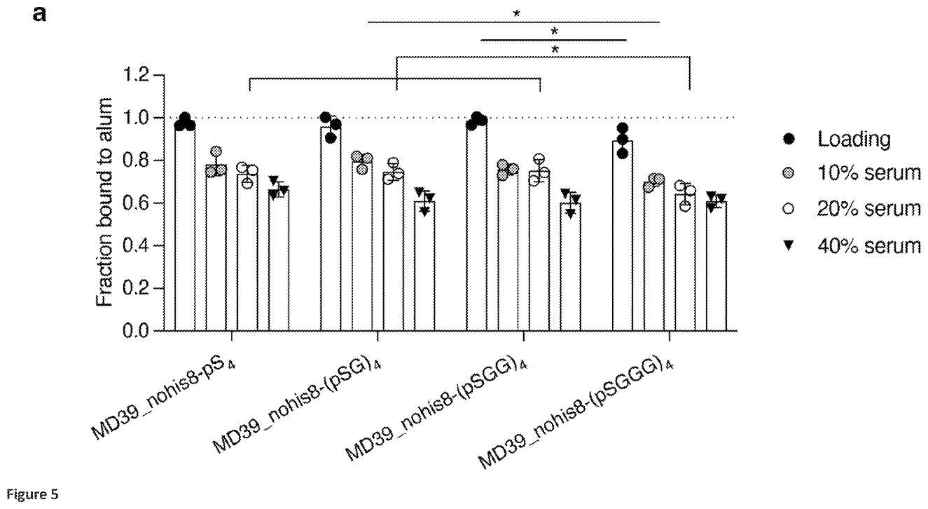

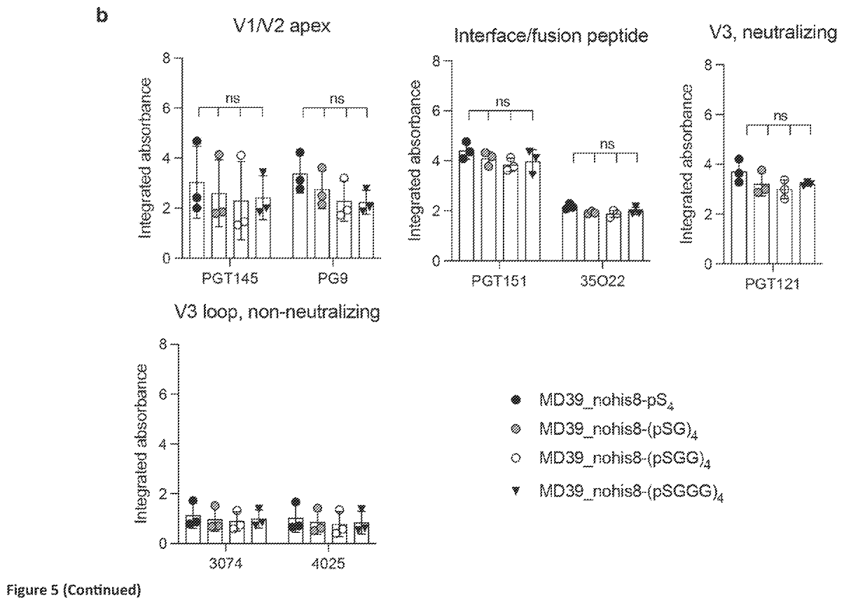

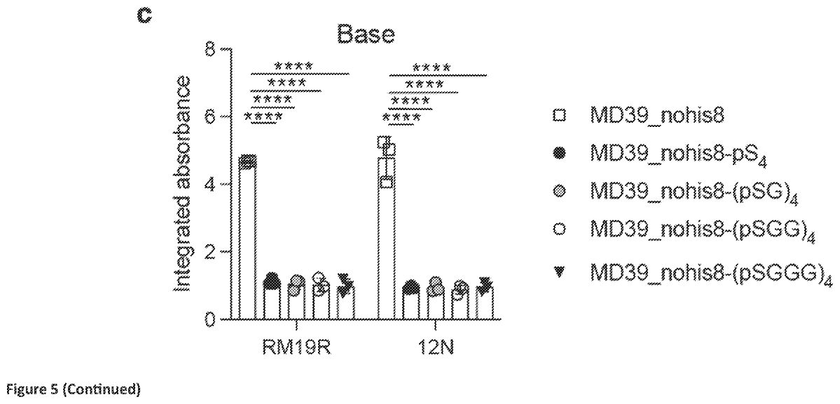

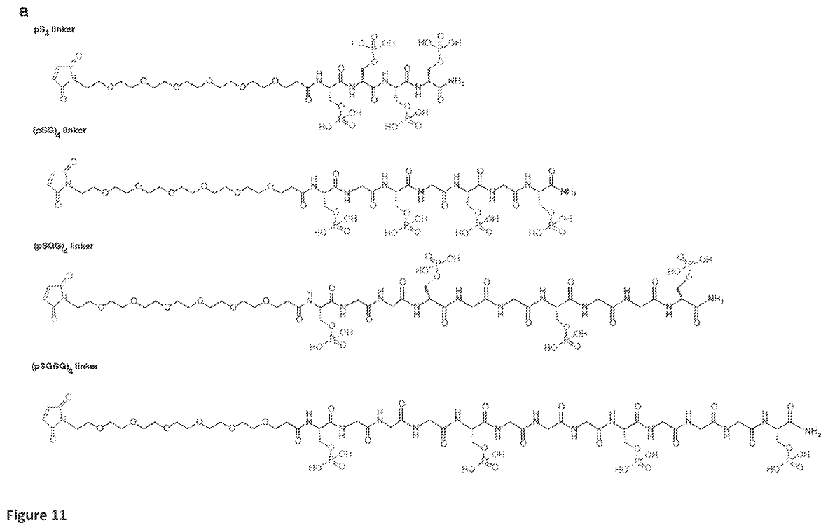

Although peptide tags comprised of 4 repeats of phosphoserine provided effective binding to alum, we hypothesized that separating phosphoserine groups with uncharged spacer residues might enable more efficient complexation of the pSer tag with the surface of alum particles and thereby further enhance the stability of trimer binding to alum. To test this idea, we screened additional pSer tags containing glycine amino acid spacers between pSer residues prepared by solid phase peptide synthesis using glycine repeats of 0 to 3 residues (pS₄, (pSG)₄, (pSGG)₄, and (pSGGG)₄; FIG. 11a). These tags were conjugated to MD39_nohis8, and the impact on alum binding and trimer antigenicity on alum was assessed. As shown in FIG. 5a, initial binding to alum in buffer was >90% for all 4 pSer tags, and they performed comparably well at promoting retention of the trimer on alum following serum exposure. Structural analysis of trimers bound to alum using neutralizing and non-neutralizing mAbs revealed a similar antigenicity profile for all trimers linked with all 4 pSer tag variants, and all 4 tags showed a similar low binding by trimer base-specific antibodies (FIG. 5b-c, 11b). Based on these findings showing no further improvement by changing the peptide sequence, we moved forward with the pSer₄ tag containing four sequential phosphoserines.

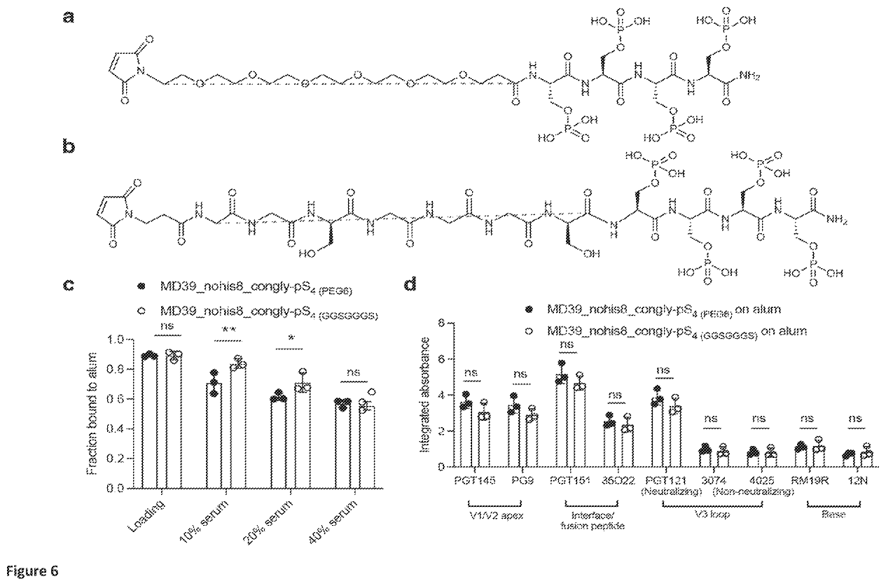

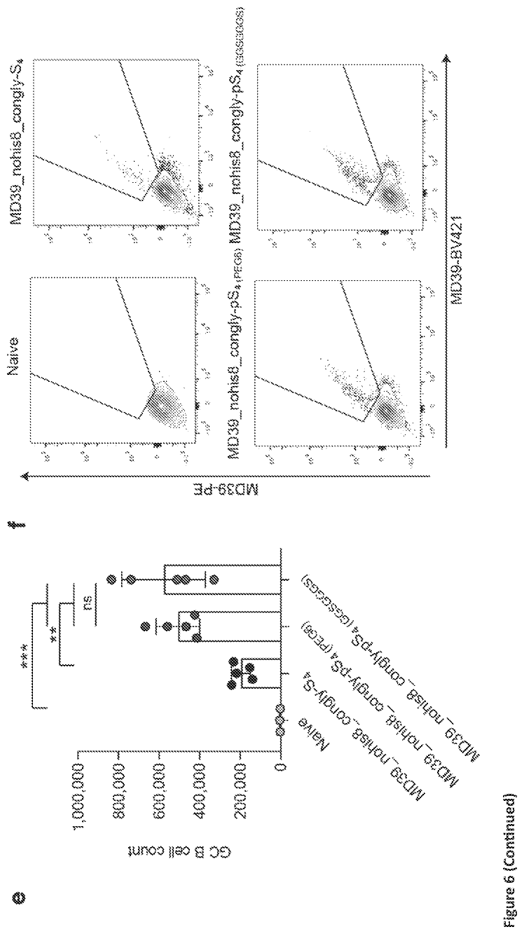

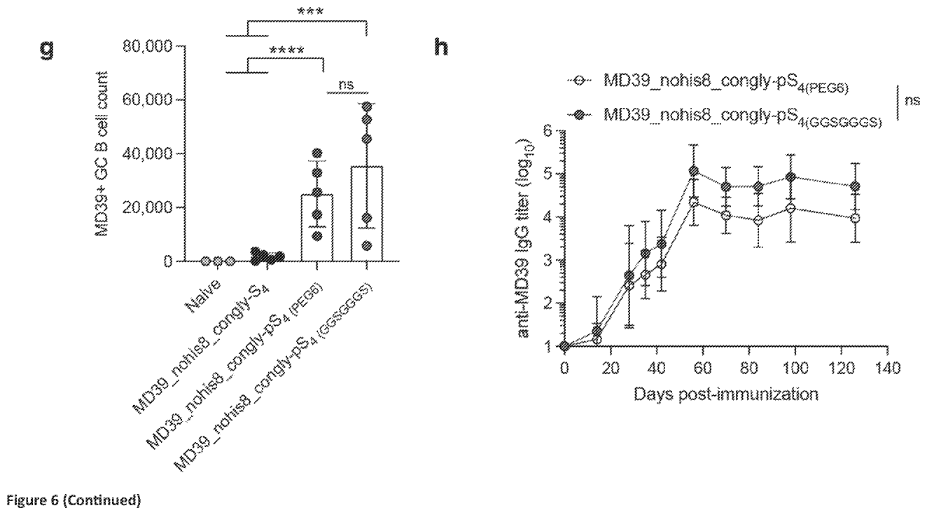

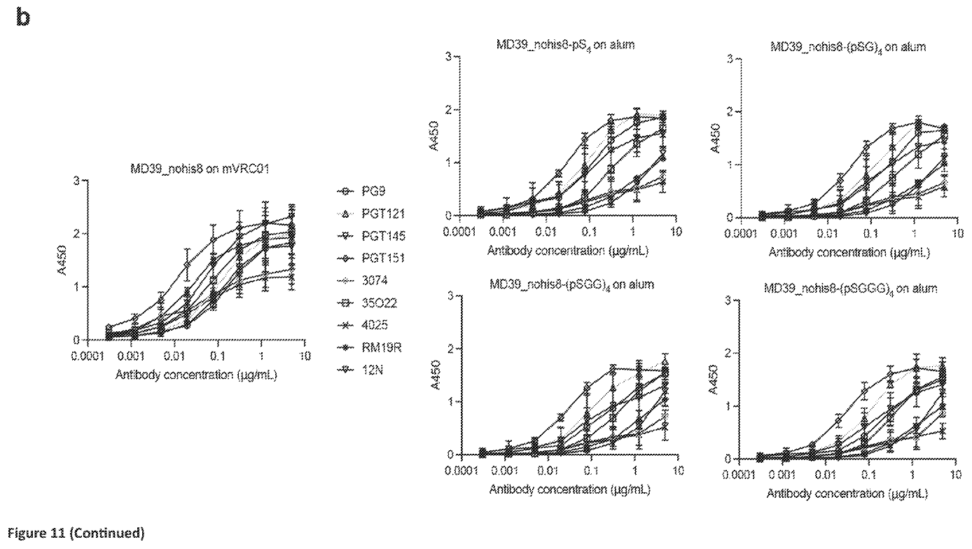

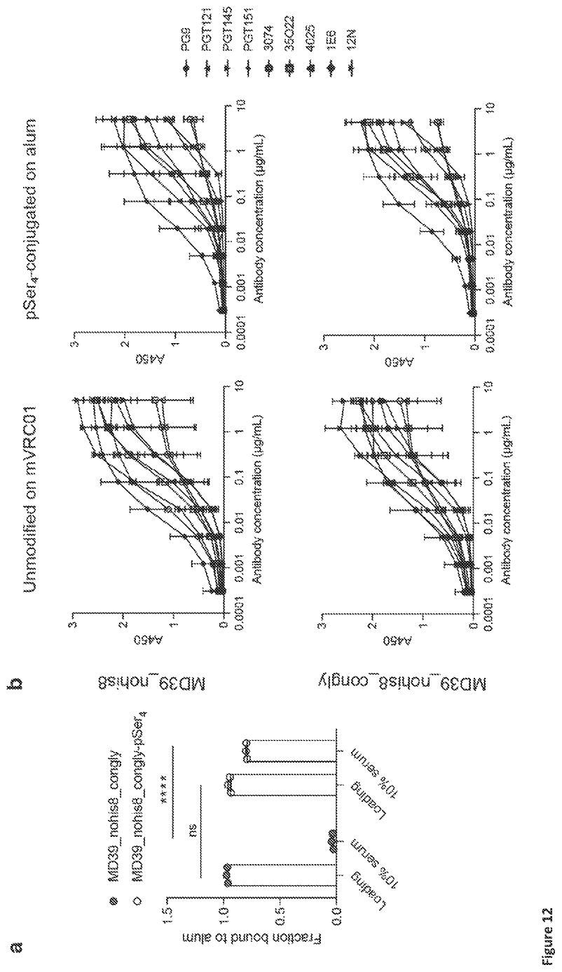

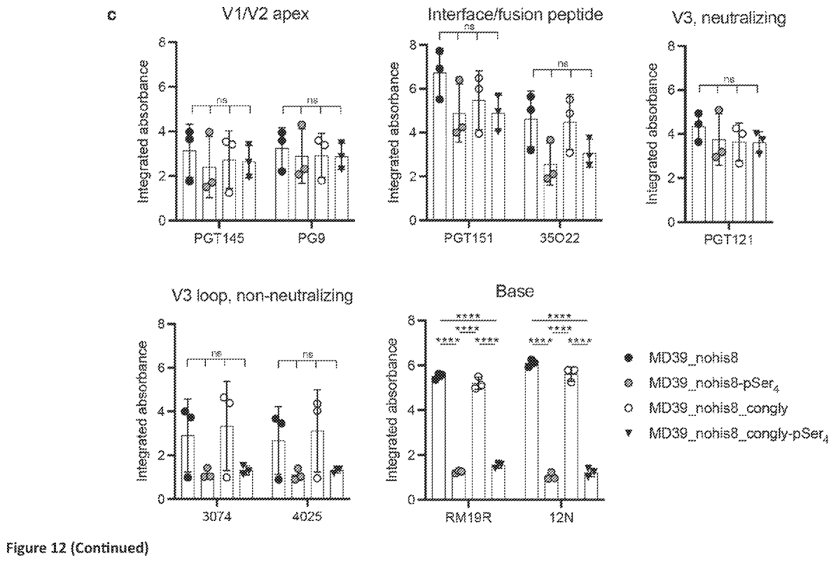

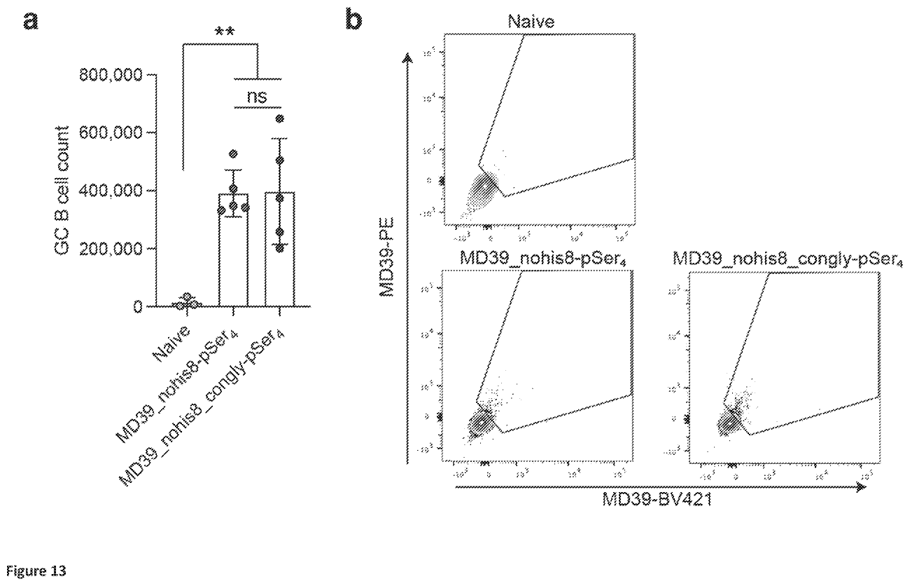

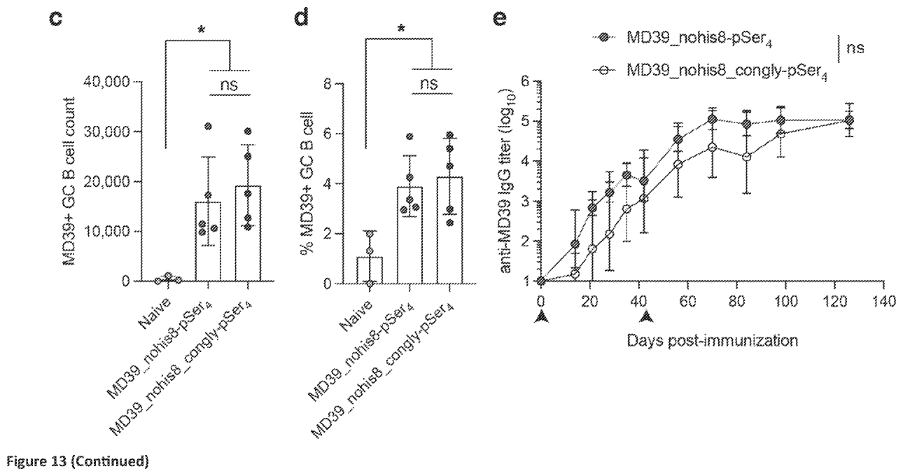

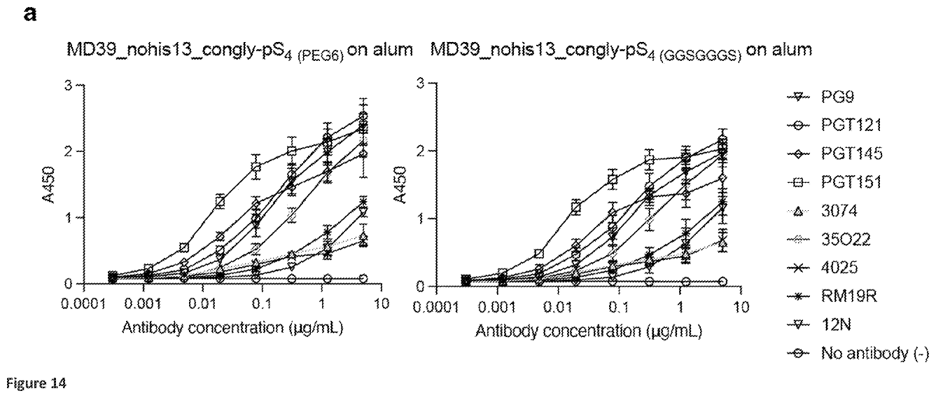

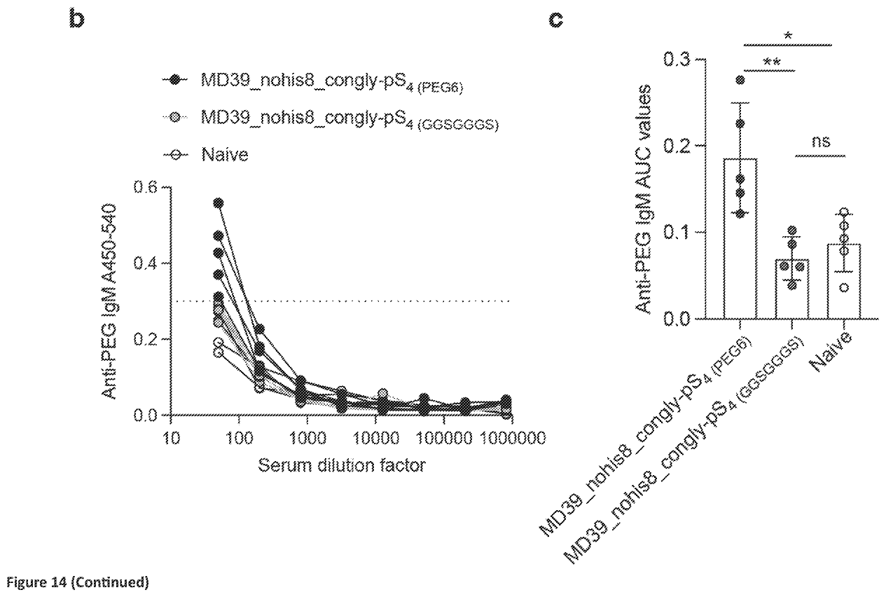

We further sought to replace the 6 unit polyethylene glycol (PEG₆) spacer present in our original pSer tag design with a short glycine/serine spacer (GGSGGGS (SEQ ID NO: 20)) of comparable fully-extended length (FIG. 6a-b), in order to reduce any potential for PEG-driven allergic reactions^{28,29} or off-target anti-PEG antibody responses, which have been previously reported for PEGylated drugs.³⁰ Prior to testing this fully peptide-based pSer₄ tag, we sought to fill an immunogenic glycan hole in the trimer immunogen at residues N241 and N289, by sequence modification to improve N4-linked glycan processing. This glycan hole is a target for HIV strain-specific neutralizing responses that cannot mature to a broadly neutralizing antibody response.^{31,32} This construct, MD39_nohis8_congly, showed the expected binding and antigenicity profile on alum when modified with phosphoserines (FIG. 12a-c). We assessed the structural integrity of the glycan hole-filled trimer on alum by antigenicity ELISA, confirming that the antigen retains major neutralizing epitopes while shielding the trimer base comparably to the glycan hole-containing construct (FIG. 12b-c). Further, GC and serum antibody responses were similar when comparing pSer₄-conjugated trimers with or without the glycan hole filled, albeit with slightly lower total serum binding antibodies for the former, as expected since the congly trimer has less exposed protein surface (FIG. 13). Based on these findings, the MD39_nohis8_congly construct was conjugated to the PEG₆ pS_{4 (PEG6)} or GGSGGGS (SEQ ID NO: 20) pS_{4 (GGGSGGGS (SEQ ID NO: 20))} tags for biochemical and immune response analysis. Interestingly, while initial alum binding of trimer modified with the two different pSer tags was essentially identical (FIG. 6c, “loading”), trimers tagged with the fully peptide spacer exhibited slightly greater retention on alum when challenged with 10 or 20% serum (FIG. 6c). Antigenicity profiling revealed no significant difference between trimers captured on alum using either tag design, with comparable shielding of the trimer base for each spacer (FIG. 6d, 14a). We then compared GC responses and serum antibody titers elicited by these two different pSer tag designs for MD39_nohis8_congly-pS₄ combined with alum and SMNP. MD39_nohis8_congly modified with the two pSer tag types elicited comparable total CC B cell counts as well as comparable numbers of MD39-specific GC B cells, and these responses were significantly stronger than responses elicited by the control MD39_nohis8_congly-S₄ trimer (FIG. 6e-g). We then compared the immunogenicity of the constructs, immunizing mice with Ser₄- or pSer₄-conjugated MD39 constructs and alum, followed by boosting with Ser₄- or pSer₄-conjugated MD39 constructs combined with alum and SMNP at 6 weeks. As shown in FIG. 6h, there was a trend toward increased serum IgG responses using the peptide-based linker vs. the PEG linker, though this did not reach statistical significance. While no anti-PEG IgG responses were detected in either group at week 8 post-prime (week 2 post-boost), there were significantly reduced anti-PEG IgM responses in the group immunized with MD39_nohis8_congly conjugated to the GGSGGGS (SEQ ID NO: 20) pSer tag (FIG. 14b-c). These data suggest that MD39 nohis8_congly-pS_{4 (GGSGGGS)(SEQ ID NO: 20)} is a favorable construct for clinical use.

A summary of certain results is provided in Tables 2-4.

TABLE 2
Data showing benefit of “nohis8” vs “his”

| Experiment/FIG. | Finding |
|---|---|
| FIG. 9a | Alum binding: |
| | MD39_nohis8-pSer₄ unexpectedly demonstrates |
| | significantly improved binding and retention on |
| | alum compared to MD39_his-pSer₄ (10%, 20%, |
| | and 40% serum conditions). |
| FIG. 3d | Antigenicity profiling: |
| | MD39_nohis8-pSer₄ demonstrates favorable, |
| | significantly reduced binding of non-neutralizing V3 |
| | loop targeting antibody 3074 and base targeting |
| | antibody RM19R compared to MD39_his-pSer₄. |
| FIG. 4a-b | Immunogenicity: |
| | MD39_nohis8-pSer₄ unexpectedly demonstrates |
| | stronger antibody responses and more consistent |
| | seroconversion in immunized mice. Additionally, |
| | these antibody responses were less base-directed |
| | (base is a non-neutralizing epitope) compared to |
| | soluble trimer demonstrated in FIG. 10b. |
| FIG. 4e-g | Germinal center B cell responses: |
| | MD39_nohis8-pSer₄ demonstrates significantly |
| | enhanced antigen-specific germinal center B |
| | responses than soluble trimer. |
| FIG. 12, 13 | Showing this works with “congly” version of |
| | MD39 (filling glycan bole at residues N241 and |
| | N289): |
| | No significant differences between MD39_nohis8- |
| | pSer₄ and MD39_nohis8_congly-pSer₄ in vitro |
| | (FIG. 12) through alum binding and antigenicity |
| | profiling. No significant differences in |
| | immunogenicity or antigen-specific germinal center |
| | B cell responses when comparing MD39_nohis8- |
| | pSer₄ and MD39_nohis8_congly-pSer₄ in mice |
| | (FIG. 13). Demonstrates potential application and |
| | relevance to other HIV Env trimers. |

TABLE 3
Supporting data showing benefit of GGSGGGS (SEQ ID NO: 20) in
pSer linker

| Experiment/Figure | Finding |
|---|---|
| FIG. 6c | Alum binding: |
| | MD39_nohis8_congly-pS_{4(GGSGGGS)(SEQ ID NO: 20)} unexpectedly |
| | demonstrates significantly improved binding and retention |
| | on alum compared to MD39_nohis8_congly-pS_{4(PEG6)} |
| | (10%, 20% serum conditions). |
| FIG. 6h, FIG. 6e-g | Immunogenicity/Germinal center B cell responses: |
| | MD39_nohis8_congly-pS_{4(GGSGGGS)(SEQ ID NO: 20)} demonstrates |
| | stronger antibody responses and germinal center B cell |
| | responses compared to the soluble antigen immunization. |
| | MD39_nobis8_congly-pS_{4(GGSGGGS)(SEQ ID NO: 20)} surprisingly |
| | trended towards higher antibody responses compared to |
| | MD39_nobis8_congly-pS_{4(PEG6)}. |
| FIG. 14b-c | Anti-PEG antibody responses: |
| | By removing the PEG6 from the pSer linker, we observed |
| | favorable, significantly reduced anti-PEG antibody |
| | responses following immunization with |
| | MD39_nohis8_congly-pS_{4(GGSGGGS)(SEQ ID NO: 20)} compared to |
| | MD39_nohis8_congly-pS_{4(PEG6)}. |

TABLE 4
Summary table of C-terminal sequences and linkers studied

| | | | | Alum binding | | |
|---|---|---|---|---|---|---|
| | | | | (% retained on | | |
| C-terminal | Antigen | pSer | Conjugation | alum with 10% | Anti- | Immuno- |
| linker | linker | linker | (~4 expected) | mouse serum) | genicity | genicity |
| GTKHHHHH | his | Maleimide- | 3.5 | 67.1% | + | + |
| H- | | PEG₆- | | | | |
| (SEQ ID NO: | | pSer₄ | | | | |
| 21)(thiol/ | | | | | | |
| maleimide | | | | | | |
| linkage)- | | | | | | |
| PEG₆-pSer₄ | | | | | | |
| GTKGHGHG | nohis | Maleimide- | 6.5 | 66.5% | ++ | |
| H- | 1 | PEG₆- | | | | |
| (SEQ ID NO: | | pSer₄ | | | | |
| 32)(thiol/ | | | | | | |
| maleimide | | | | | | |
| linkage)- | | | | | | |
| PEG₆-pSer₄ | | | | | | |
| GTKHHHH- | nohis | Maleimide- | 5.4 | 71.9% | + | |
| (SEQ ID NO: | 2 | PEG₆- | | | | |
| 33)(thiol/ | | pSer₄ | | | | |
| maleimide | | | | | | |
| linkage)- | | | | | | |
| PEG₆-pSer₄ | | | | | | |
| GTKGHGK- | nohis | Maleimide- | 5.4 | 71.0% | ++ | |
| (SEQ ID NO: | 3 | PEG₆- | | | | |
| 34)(thiol/ | | pSer₄ | | | | |
| maleimide | | | | | | |
| linkage)- | | | | | | |
| PEG₆-pSer₄ | | | | | | |
| GTKKKKK- | nohis | Maleimide- | 6.2 | 88.2% | +++ | + |
| (SEQ ID NO: | 4 | PEG₆- | | | | |
| 35)(thiol/ | | pSer₄ | | | | |
| maleimide | | | | | | |
| linkage)- | | | | | | |
| PEG₆-pSer₄ | | | | | | |
| GTKHHH- | nohis | Maleimide- | 7.5 | 70.4% | ++ | |
| (SEQ ID NO: | 5 | PEG₆- | | | | |
| 36)(thiol/ | | pSer₄ | | | | |
| maleimide | | | | | | |
| linkage)- | | | | | | |
| PEG₆-pSer₄ | | | | | | |
| GTKKKK- | nohis | Maleimide- | 10.4 | 88.0% | ++ | |
| (SEQ ID NO: | 6 | PEG₆- | | | | |
| 37)(thiol/ | | pSer₄ | | | | |
| maleimide | | | | | | |
| linkage)- | | | | | | |
| PEG₆-pSer₄ | | | | | | |
| GTKHH- | nohis | Maleimide- | 4.9 | 66.6% | ++ | |
| (SEQ ID NO: | 7 | PEG₆- | | | | |
| 38) (thiol/ | | pSer₄ | | | | |
| maleimide | | | | | | |
| linkage)- | | | | | | |
| PEG₆-pSer₄ | | | | | | |
| GTKKK- | nohis | Maleimide- | 4.9 | 82.8% | +++ | ++ |
| (SEQ ID NO: | 8 | PEG₆- | | | | |
| 3)(thiol/ | | pSer₄ | | | | |
| maleimide | | | | | | |
| linkage)- | | | | | | |
| PEG₆-pSer₄ | | | | | | |
| GTKEEE- | nohis | Maleimide- | 4.6 | 52.1% | − | |
| (SEQ ID NO: | 9 | PEG₆- | | | | |
| 39) | | pSer₄ | | | | |
| (thiol/ | | | | | | |
| maleimide | | | | | | |
| linkage)- | | | | | | |
| PEG₆-pSer₄ | | | | | | |
| GTKKK- | nohis | Maleimide- | Consistent | 83.8% | +++ | +++ |
| (SEQ ID NO: | 8 | GGSGGGS- | with | (MD39_nohis8_ | | |
| 3)(thiol/ | | pSer₄ | GTKKK- | congly with | | |
| maleimide | | (SEQ ID | (SEQ ID NO: | maleimide- | | |
| linkage)- | | NO: 20) | 3) (thiol/ | GGSGGGS- | | |
| GGSGGGS- | | | maleimide | pSer₄ (SEQ ID | | |
| pSer₄ (SEQ ID | | | linkage)- | NO: 20) is | | |
| NO: 20) | | | PEG₆-pSer₄ | significantly | | |
| (+/− congly) | | | | better than | | |
| | | | | MD39_nohis8_ | | |
| | | | | congly with | | |
| | | | | maleimide- | | |
| | | | | PEG₆-pSer₄ | | |

### Discussion

Recent studies have demonstrated an important role for the timing of antigen and adjuvant. delivery to lymph nodes during immunization, with prolonged vaccine exposure over ~2-4 weeks primming enhanced humoral immunity compared to bolus immunization in both mice and non-human primates. We recently developed an approach to achieve “extended dosing” effects using the traditional adjuvant alum by modifying antigens with a short tag containing pSer residues which anchor the antigen to alum through ligand exchange.

Here we carried out a systematic study evaluating design parameters intrinsic to the pSer-antigen/alum approach, with the goal of providing an improved stabilized trimer design to maximally elicit on-target, vaccine-relevant responses. We tested the importance of pSer valency immunogen linkers, and the composition of the pSer peptide tag, and employed an alum-binding co-adjuvant SMNP to synergistically boost humoral immune responses. The phosphate valency of pSer₄ is sufficient to enhance humoral immune responses. We similarly observed a reduction in responses for pSer₈ which may be due to altered antigen drainage kinetics. Interestingly, replacing; the polyhistidine linker at the C-terminus of the trimer with a positively charged amino acid sequence significantly improved alum binding strength and trimer base shielding with improved immunogenicity in mice when conjugated to pSer₄. Inter-pSer glycine spacer residues did not have a significant effect, suggesting the flexibility of the peptide promotes sufficient access of pSer phosphates to hydroxyls on the surface of alum for ligand exchange. Unexpectedly, we observed an improvement in immune responses when the PEG₆ linker in the pSer tag was replaced with a GGSGGGS (SEQ ID NO: 20) amino acid sequence, This may be due to alterations in the interaction of the linker sequence with the surface of alum particles, or subtle changes in the linker organization at the trimer base. This improved MD39-pSer/alum approach elicited robust antigen-specific GC B cell and serum IgG responses, maximizing humoral responses and minimizing off-target, vaccine-irrelevant responses to non-neutralizing epitopes.

Here, we systematically modulated and evaluated parameters relevant to both the alum-binding tag and antigen, including phosphate valency and spacing, spacer sequence, and antigen linker design. Such modifications improved vaccine-elicited immune responses and help to advance this platform toward clinical use.

In conclusion, through facile modification of immunogens with a short peptide linker, we observed striking enhancements in antibody and GC responses using alum, a well-established adjuvant, to achieve extended dosing in vaccination to prolong antigen availability to achieve stronger and more durable GC responses. This platform transforms alum, a low-cost FDA-approved vaccine adjuvant with a long clinical record of safety and modest efficacy when mixed with immunogens, into a more efficacious clinically relevant platform applicable to the development of vaccines targeting other established and emerging infectious diseases and cancer. Through co-anchoring of additional adjuvants, this approach may provide synergistic combinations of adjuvants for vaccine use.

### Example 2. Germline-Targeting Trimers, Including N332-GT5

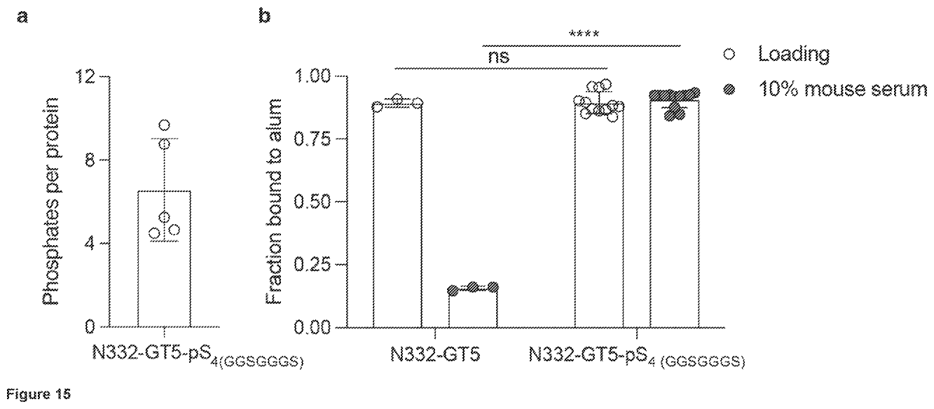

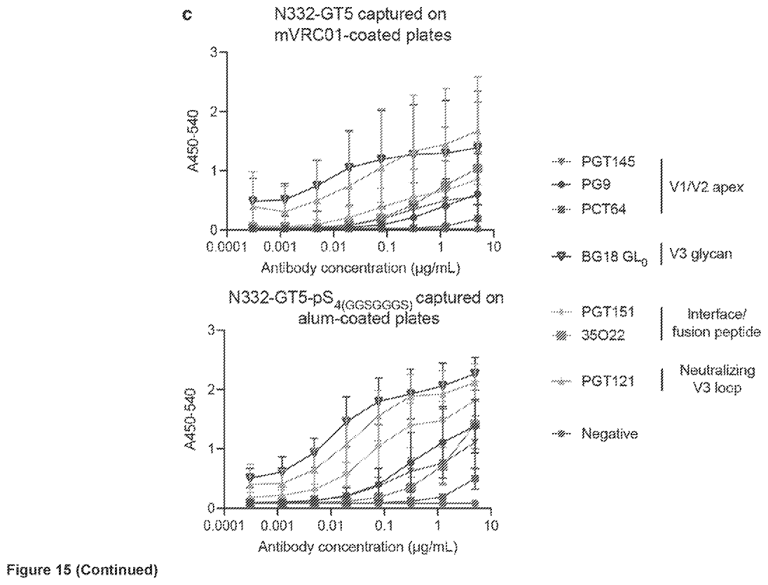

N332-GT5 with nohis8 was conjugated to maleimide-GGSGGGS-pSer_{4 (SEQ ID NO: 22)}, and phosphate analysis indicated successful pSer tag conjugation (FIG. 15a). We then assessed the stability of alum binding for-N332-GT5-pS_{4 (GGSGGGS)(SEQ ID NO: 20)}. Trimers were incubated with alum for 30 minutes in tris-buffered saline to allow adsorption to the alum particles, followed by incubation for 24 hours in 10% mouse serum at 37° C. to test binding stability. Similar to findings with MD39, unmodified N332-GT5 showed poor retention on alum in the presence of 10% mouse serum while in comparison, the N332-GT5-pS_{4(GGSGGGS) (SEQ ID NO: 20)} exhibited significantly higher and near-complete retention on alum (FIG. 15b). To confirm that N332-GT5-pS_{4(GGSGGGS) (SEQ ID NO: 20)} retained structural integrity upon binding to alum, we probed the antigenicity profiles of the constructs, Antigens were captured on murine VRC01- or alum-coated plates, and the immobilized immunogen was probed for binding to serial dilutions of a panel of neutralizing and non-neutralizing monoclonal antibodies. This analysis indicated comparable antigenicity profiles for N332-GT5-pS_{4(GGSGGGS)(SEQ ID NO: 20)} captured on alum-coated ELISA plates vs. unmodified N332-GT5 captured on murine VRC01 mAb-coated plates (FIG. 15c). This suggests that this improved approach is compatible with germline targeting immunogens, including N332-GT5.

### Methods

### Phosphoserine Peptide Synthesis

pSer peptide linkers were synthesized using solid phase synthesis on low-loading TentaGel™ Rink Amide resin (0.2 meq/g, Peptides International) as described previously. Briefly, resin was deprotected with 20% piperidine (Sigma Aldrich) in dimethylformamide (DMF Sigma Aldrich), and peptide couplings were performed with 4 equivalents of Fmoc-Ser(PO(OBzl)OH)-OH (Millipore Sigma) and 3.95 equivalents of hexafluorophosphate azabenzotriazole tetramethyl uranium (HATU) for 2 hours at 25° C. in DCM and DMF (1:2). pSer residues were deprotected with 5% 1,8-diazabicyclo[5.4.0]undec-7-ene (DBU) in DMF. Double couplings were performed after the third residue. An Fmoc-protected 6-unit oligoethylene glycol linker (Peptides International) was then coupled to the peptide and subsequently deprotected and reacted with N-maleoyl-β-alanine (Sigma Aldrich). Completion of each deprotection and coupling step was confirmed by a ninhydrin test (Sigma Aldrich). pSer side chains were deprotected and the peptide was cleaved from the resin in 95% trifluoroacetic acid (Sigma Aldrich), 2.5% H₂0, and 2.5% triisopropylsilane (Sigma Aldrich), for 2.5 hours at 25° C. The product was precipitated in 4° C. diethyl ether (Sigma Aldrich) and dried under N2, then purified by HPLC on a C18 column (Agilent Zorbax 300SB-C18) using 0.1 M triethylammonium acetate buffer (Glen Research) in an acetonitrile gradient. The peptide mass was confined by matrix-assisted laser desorption/ionization-time of flight mass spectrometry.

For imaging experiments, a pSer₄-AlexaFluor™488 conjugate was synthesized as described for the pSer component of the linker, followed by deprotection and coupling to Fmoc-5-azido-pentanoic acid (Anaspec). The peptide was deprotected with 20% piperidine in dimethylfomamide prior to cleavage from the resin in 95% trifluoroacetic acid, 2.5% H₂0, and 2.5% triisopropylsilane for 2.5 hours at 25° C. The product was then precipitated in 4° C. diethyl ether, and dried under N2, and purified by HPLC on a C18 column using 0.1M triethylammonium acetate buffer in an acetonitrile gradient. The peptide mass was confirmed by matrix-assisted laser desorption/ionization-time of flight mass spectrometry. This pSer₄-azide linker was reacted with one equivalent of AlexaFluor488-DBCO (Click Chemistry Tools) overnight at 4° C. in a Cu-free click reaction in PBS (pH 7.2-7.4) and subsequently purified by HPLC on a C18 column using 0.1M triethylammonium acetate buffer in an acetonitrile gradient. The pSer peptide tags containing glycine spacers between pSer residues and glycine-glycine-serine-glycine-glycine-glycine-serine in place of the poly(ethylene glycol) spacer were prepared by solid phase peptide synthesis by Almac and characterized by HPLC and mass spectrometry.

Antigen Production and pSer Conjugation

MD39 immunogens were synthesized as described previously.^{21,40} Briefly, genes encoding MD39 immunogens were cloned into pHLsec by Genscript and co-transfected with human furin in a pcDNA3.1 plasmid using a 2:1 trimer:furin DNA ratio with polyethylenimine into FreeStyle™ 293-F cells (Thermofisher) and incubated for 6 days. The cultures were centrifuged and the supernatants containing MD39 were harvested and purified using a HisTrap HP column (Cytiva Life Sciences) with an AKTA™ FPLC system (Cytiva Life Sciences) for immunogens expressed with a polyhistidine linker and a 2G 12 immunoaffinity column for MD39 immunogens without a polyhistidine linker. The immunogens were further purified by size-exclusion chromatography with an S200 Increase column (Cytiva Life Sciences) in TBS at flow rate of 0.5 ml/min. Size exclusion chromatography multi-angle light-scattering (SECMALS, DAWN HELEOS II and Optilab T-rEX™ Wyatt Technology) was then used to confirm the immunogen molecular weight.

Immunogens expressed with a free terminal cysteine were reduced at 1 mg/ml with 10 molar equivalents of tris(2-carboxyethyl)phosphine (TCEP, ThermoFisher) and incubated at 25° C. for 10 minutes. TCEP was subsequently removed from reduced protein solutions using Amicon™ Ultra Centrifugal Filters (10 kDa MWCO, Millipore Sigma) in tris-buffered saline (TBS, Sigma Aldrich), and 1 mg/ml antigen was reacted with 5 molar equivalents of pSer-maleimide linkers for 16 hours at 4° C. in TBS (pH 7.2-7.4). Free pSer linker was subsequently removed using centrifugal filters in TBS, and pSer-antigen was buffer exchanged to PBS.

pSer₄-conjugated cytochrome C used for antigenicity profiling of immunogens was prepared as described,¹⁸ using cytochrome C from Saccharomyces cerevisiae (Sigma Aldrich). The number of pSer residues conjugated to the antigen was assessed using the Malachite™ Green Phosphoprotein Phosphate Estimation Assay Kit (Thermo Scientific) against a standard curve of pSer-maleimide linker. Signal from pSer-antigen was compared to the background from an unconjugated antigen control. Fluorescently labeled protein used in imaging experiments were prepared by reacting 1 mg/ml antigen in 50 mM sodium bicarbonate buffer for 1 hour at 25° C. with 6 molar equivalents of AlexaFluor™647 NHS ester (Invitrogen) for alum binding studies and whole-mouse imaging. Labeled antigen was purified by centrifugal filtration.

### SMNP Adjuvant Synthesis

Saponin-MPLA nanoparticles (SMNP) adjuvant was prepared as previously described.²⁴ Briefly, solutions at 20 mg/ml were prepared of cholesterol, DPPC, and PHAD MPLA (Avanti Polar Lipids) in 20% MEGA-10 detergent (Sigma). Quil-A® saponin (InvivoGen) was dissolved in Milli-QM water at a final concentration of 100 mg/ml. These were mixed at a mass ratio of 10:2:1:1 (Quil-A:chol:DPPC:MPLA) and diluted in PBS to a final cholesterol concentration of 1 mg/ml. The solution was equilibrated overnight at 25° C. and then dialyzed against PBS using a 10 kDa MWCO cassette. The adjuvant was then sterile filtered, concentrated using Amicon™ Ultra Centrifugal Filters (50 kDa MWCO, Millipore Sigma), and purified by FPLC using a Sephacryl™ S-500 HR size exclusion column (Cytiva Life Sciences). The concentration was determined using a cholesterol quantification assay (Sigma Aldrich).

### Antigen and Adjuvant Alum Binding and Release

AlexaFluor™647-labeled antigen was loaded onto Alhydrogel™ (alum, InvivoGen) in TBS at a 1:10 antigen:alum mass ratio, unless otherwise specified, for 30 minutes on a tube rotator at 25° C. To assess antigen binding to alum, samples were immediately centrifuged at 10,000×g for 10 minutes to pellet alum, and the fluorescence of the supernatant was measured against a standard curve of labeled antigen. To assess the release of antigen from alum, mouse serum was added to antigen-alum solutions post-loading to a final mouse serum concentration of 10 vol % and incubated at 37° C. for 24 hours, unless otherwise specified. Samples were subsequently centrifuged at 10,000 g for 10 minutes to pellet alum, and the fraction of protein bound to alum was measured by fluorescence analysis of the supernatant using a Tecan Infinite M200 Pro plate reader Experiments investigating SMNP binding and release from alum were performed using Cy7-labeled SMNP, prepared as described incorporating 1,2-distearoyl-sn-glycero-3-phosphoethanolamine-N-(Cy7) (Avanti Polar Lipids) in place of 10 mol % of the MPLA.

### Antigenicity Profiling of Immunogens

Antigenicity profiling of antigens was completed by comparing antibody binding curves of pSer-conjugated MD39 on alum against those of unmodified MD39. To capture alum on Nunc Maxisorp™ ELISA plates (Invitrogen), plates were first coated with pSer₄-conjugated cytochrome C at 2 μg/ml for 4 hours at 25° C. Alum was then added at 200 μg/ml and captured by pSer₄-cytochrome C for 16 hours at 4° C. To capture unmodified MD39 with a polyhistidine linker, plates were coated with a rabbit anti-histag antibody (GenScript) at 2 μg/ml for 4 hours at 25° C. followed by blocking with 2% BSA in PBS for 16 hours at 4° C. To capture unmodified MD39 expressed with non-polyhistidine linkers, plates were coated with mouse VRC01 at 2 μg/ml for 4 hours at 25° C. followed by blocking with 2% BSA in PBS for 16 hours at 4° C. Plates were washed with 0.05% Tween-20 in PBS and incubated with 2 μg/ml protein in 2% BSA in PBS for 2 hours at 25° C. Neutralizing and non-neutralizing antibodies were added at 5 μg/ml with 1:4 serial dilutions for 2 hours at 25° C. Plates were washed and antibody binding was detected with a goat anti-human HRP conjugated secondary antibody with minimal cross-reactivity (Jackson ImmunoResearch) at 1:5000 dilution in PBS containing 2% BSA and then developed with 3,3′,5,5′-tetramethylbenzidine (ThermoFisher), stopped with 2N sulfuric acid and immediately read (450 nm with 540 nm reference) on a BioTek Synergy2 plate reader.

### Animals and Immunizations

Experiments and handling of mice were conducted under federal, state and local guidelines under an Institutional Animal Care and Use Committee (IACUC) approved protocol. Female 6-8-week-old BALB/c mice were purchased from the Jackson Laboratory (stock no. 000651). Immunizations were prepared by mixing 10 μg of antigen and 100 μg of alum in 100 μL sterile tris-buffered saline (TBS, Sigma Aldrich) per mouse, unless otherwise specified. Antigen was loaded onto alum for 30 minutes on a tube rotator prior to immunization. When SMNP was added into the immunization, antigen was first loaded onto alum for 30 minutes on a rotator, after which 5 tig of SMNP unless otherwise specified was added into the immunization and incubated with antigen-alum formulations for 30 minutes prior to immunization. This dose of SMNP corresponds to 5 μg of Quil-A and 0.5 μg MPLA. Mice were immunized subcutaneously at the tail base with 50 μL on each side of the tail base and were subsequently boosted 6 weeks post-prime.

### Antigen-Binding ELISA

Serum was collected from mice retro-orbitally using capillary tubes and stored at −20° C. until analysis. To determine serum IgG titers, Nunc Maxisorp™ plates (Invitrogen) were coated with a rabbit anti-histag antibody (GenScript) at 2 μg/ml for 4 hours at 25° C. in PBS and blocked with 2% BSA in PBS overnight at 4° C. Plates were washed with 0.05% Tween-20 PBS, and MD39 was added at 2 μg/ml in 2% BSA in PBS for 2 hours. Serum dilutions (1:10 dilution followed by 1:50 dilution with 1:4 serial dilutions) were incubated in the plate for 2 hours. Plates are washed again, incubated with a goat anti-mouse IgG HRP-conjugated secondary (BioRad) at 1:5000 dilution, and then developed with 3,3′,5,5′-tetramethylbenzidine (ThermoFisher), stopped with 2N sulfuric acid, and immediately read (450 nm with 540 nm reference) on a BioTek Synergy2™ plate reader. To determine non-trimer base-directed serum IgG titers, Nunc Maxisorp plates (Invitrogen) were coated with a rabbit anti-histag antibody (GenScript) at 2 μg/ml for 4 hours at 25° C. in PBS and blocked with 2% BSA in PBS overnight at 4° C., and then incubated for 30 minutes with base-binding 12N antibody. Serum dilutions (1:10 dilution followed by 1:50 dilution with 1:4 serial dilutions) were then added to the plate for 2 hours, and subsequently follow the protocol for anti-MD39 serum IgG titers. For studies comparing proteins with varying linkers, Nunc Maxisorp™ plates (Invitrogen) were coated with human VRC01 at 2 μg/ml for 4 hours at 25° C. in PBS and blocked with 2% BSA in PBS overnight at 4° C. Plates were washed with 0.05% Tween-20 PBS, and MD39 with a linker orthogonal to the immunization constructs was added at 2 μg/ml in 2% BSA in PBS for 2 hours, and subsequently follow the protocol for anti-MD39 serum IgG titers. To determine anti-PEG serum responses, Nunc Maxisorp™ plates (Invitrogen) were coated with streptavidin (Invitrogen) at 1 μg/mL for 4 hours at 25° C. in PBS and blocked with 2% BSA in PBS overnight at 4° C. Plates were washed with 5% (w/v) n-Dodecyl-beta-Maltoside (ThermoFisher) in PBS, and biotin-PEG-OH (MW 2k, Creative PEGWorks) was added at 1 μg/mil in 2% BSA in PBS for 2 hours. Serum dilutions (1:50 dilution followed by 1:4 serial dilutions) were incubated in the plate for 2 hours. Plates are washed again, incubated with a goat anti-mouse IgG or IgM IRP-conjugated secondary (BioRad) at 1:5000 dilution, and then developed with 3,3′,5,5′-tetramethylbenzidine (ThermoFisher), stopped with 2N sulfuric acid, and immediately read (450 nm with 540 nm reference) on a BioTek Synergy 2™ plate reader.

### Germinal Center and T Follicular Helper Responses

The inguinal lymph nodes were collected from immunized mice 14 days post-immunization unless otherwise specified. For germinal center analysis, cells were stained for viability (Thermofisher Live/Dead Fixable Aqua) and against CD3e (BV711, BioLegend, 145-2C11 clone), B220 (PE-Cy7, BioLegend RA3-6B2 clone), CD38 (FITC, BioLegend 90 clone), and GL7 (PerCP-Cy5.5, BioLegend GL7 clone), with antigen-specific staining completed using biotinylated MD39 conjugated to streptavidin-BV421 (BioLegend) and streptavidin-PE (BioLegend). Samples were analyzed by flow cytometry on a BD Celesta and analyzed on FlowJo.

### Whole-Mouse Imaging of Vaccination Drainage

Mice were immunized subcutaneously at the tail base with fluorescently labeled antigen. Immunizations were prepared as described, using fluorescently labeled components as indicated. For studies including fluorescently labeled components, immunizations were prepared by loading antigen onto alum in sterile tris-buffered saline (TBS, Sigma Aldrich) for 30 minutes on a tube rotator prior to adding SMNP if indicated and incubating for 30 minutes on a tube rotator. Imaging was completed using a PerkinElmer Xenogen™ Spectrum vivo imaging system (IVIS), and the fluorescent signal at the injection site was quantified using LivingImage software. The radiant efficiency was tracked longitudinally to monitor drainage from the injection site.

### Statistical Analysis

All data were plotted and all statistical analyses were performed using GraphPad Prism 9 software (La Jolla, CA). All graphs display mean values, and the error bars represent the standard deviation unless otherwise specified. No samples or animals were excluded from the analyses. Statistical comparison was performed using a one-way ANOVA followed by Tukey's post-hoc test for single timepoint data and two-way ANOVA followed by Tukey's post-hoc test for multi-timepoint longitudinal data. Statistical analysis of antibody titer was completed using log-transformed data. Data were considered statistically significant if the p-value was less than 0.05.

### REFERENCES

1. (UNAIDS), J. U. N. P on H. and A. Global HIV & AIDS statistics. unaids.org/en/resources/fact-sheet#:~:text=GLOBAL%20HIV%20STATISTICS&text=37.7%20million%20%5B30.2%20million%E2%80%9345.1,AIDS%2Drelated%20illnesses%20in%202020.
2. Fauci, A. S. An HIV Vaccine is Essential for Ending the HIV/AIDS Pandemic. Jama 318, 1535 (2017).
3. Rerks-Ngarm, S. et al. Vaccination with ALVAC and AIDSVAX to Prevent HIV-1 Infection in Thailand. New Engl J Med 361, 2209-2220 (2009).
4. Pollara, J., Easterhoff, D. & Fouda, G. G. Lessons learned from human HIV vaccine trials. Curr Opin Hiv Aids 12, 216-221 (2017).
5. Burton, D. R. & Hangartner, L. Broadly Neutralizing Antibodies to HIV and Their Role in Vaccine Design. Annu Rev Immunol 34, 635-659 (2016).
6. Nishimura, Y. & Martin, M. A. Of Mice. Macaques, and Men: Broadly Neutralizing Antibody Immunotherapy for HIV-1. Cell Host Microbe 22, 207-216 (2017).
7. Corey, L. et al. Two Randomized Trials of Neutralizing Antibodies to Prevent HIV-1 Acquisition. New Engl J Med 384, 1003-1014 (2021).
8. Burton, D. R. Advancing an HIV vaccine; advancing vaccinology. Nat Rev Immunol 19, 77-78 (2019).
9. Klein, F. et al. Somatic Mutations of the Immunoglobulin Framework Are Generally Required for Broad and Potent HIV-1 Neutralization. Cell 153, 126-138 (2013).
10, Kwong, P, D. & Mascola, J. R. Human Antibodies that Neutralize HIV-1: Identification. Structures, and B Cell Ontogenies. Immunity 37, 412-425 (2012).
11. Tam, H. H. et al. Sustained antigen availability during germinal center initiation enhances antibody responses to vaccination. Proc National Acad Sci 113, E6639-E6648 (2016).
12. Cirelli, K, M. et al. Slow Delivery Immunization Enhances HIV Neutralizing Antibody and Germinal Center Responses via Modulation of Immunodominance. Cell 177, 1153-1171.e28 (2019).
13. Jelley-Gibbs, D. M. et al. Unexpected prolonged presentation of influenza antigens promotes CD4 T cell memory generation. J Exp Medicine 202, 697-706 (2005).
14. Pauthner, M. et al. Elicitation of Robust Tier 2 Neutralizing Antibody Responses in Nonhuman Primates by HIV Envelope Trimer Immunization Using Optimized Approaches. Immunity 46, 1073-1088.e6 (2017).
15. Lee, J. H. et al. Long-primed germinal centres with enduring affinity maturation and clonal migration. Nature 609, 998-1004 (2022).
16. Morefield, G. L. et al. Effect of phosphorylation of ovalbumin on adsorption by aluminum-containing adjuvants and elution upon exposure to interstitial fluid. Vaccine 23, 1502-1506 (2005).
17. Iyer, S., HogenEsch, H. & Hem, S. L. Relationship between the degree of antigen adsorption to aluminum hydroxide adjuvant in interstitial fluid and antibody production. Vaccine 21, 1219-1223 (2003).
18. Mover. T. J. et al. Engineered immunogen binding to alum adjuvant enhances humoral immunity. Nat Med 26, 430-440 (2020).
19. Rodrigues, K. A. et al. Phosphate-mediated coanchoring of RBD immunogens and molecular adjuvants to alum potentiates humoral immunity against SARS-CoV-2. Sci Adv 7, eabj6538 (2021).
20. Steichen, J. M. et al. HIV Vaccine Design to Target Germline Precursors of Glycan-Dependent Broadly Neutralizing Antibodies. Immunity 45, 483-496 (2016),
21. Kulp, D. W. et al. Structure-based design of native-like HIV-1 envelope trimers to silence non-neutralizing epitopes and eliminate CD4 binding. Nat Commun 8, 1655 (2017).
22, Andrabi, R., Bhiman, J. N. & Burton, D. R. Strategies for a multi-stage neutralizing antibody-based HIV vaccine. Curr Opin Innmunol 53, 143-151 (2018).
23 Watson, D. S., Platt, V. M., Cao, L., Venditto, V. 1. & Szoka, F. C. Antibody Response to Polyhistidine-Tagged Peptide and Protein Antigens Attached to Liposomes via Lipid-Linked Nitrilotriacetic Acid in Mice. Clin Vaccine Immunol 18, 289-297 (2011).
24. Silva, M. et al. A potent saponin and TLR agonist particulate vaccine adjuvant alters lymphatic flow and lymph node antigen accumulation. Science Immunology 6, (2021).
25. Havenar-Daughton, C., Lee, J. H. & Crotty, S. Tfh cells and HIV bnAbs, an immunodominance model of the HIV neutralizing antibody generation problem. Immunol Rev 275, 49-61 (2017).
26. Luo, Q., Tao, Y., Sheng, W., Lu, J. & Wang, H. Dinitroimidazoles as bifunctional bioconjugation reagents for protein functionalization and peptide macrocyclization. Nat Commun 10, 142 (2019).
27. Zhang, Y. et al. Thiol Specific and Tracelessly Removable Bioconjugation via Michael Addition to 5-Methylene Pyrrolones. J Am. Chem Soc 139, 6146-6151 (2017).
28. Kelso, J. M. Anaphylactic reactions to novel mRNA SARS-CoV-2/COVID-19 vaccines. Vaccine 39, 865-867 (2021).
29. Castells, M. C. & Phillips, E. J. Maintaining Safety with SARS-CoV-2 Vaccines. New Engl J Med 384, 643-649 (2020). 30 Wang, X., Ishida, T. & Kiwada, H. Anti-PEG IgM elicited by injection of liposomes is involved in the enhanced blood clearance of a subsequent dose of PEGylated liposomes. J Control Release 119, 236-244 (2007).
31. Klasse, P. J. et at. Epitopes for neutralizing antibodies induced by HIV-1 envelope glycoprotein B3505 SOSIP trimers in rabbits and macaques. Plos Pathog 14, e1006913 (2018).
32. McCoy, L. E. et al. Holes in the Glycan Shield of the Native HIV Envelope Are a Target of Trimer-Elicited Neutralizing Antibodies. Cell Reports 16, 2327-2338 (2016).
33 Stinson, J. A. et al. Enhancing influenza vaccine immunogenicity and efficacy through infection mimicry using silk microneedles. Vaccine 39, 5410-5421 (2021).
34. Roth, G. A. et at. Prolonged Codelivery of Hemagglutinin and a TLR7/8 Agonist in a Supramolecular Polymer-Nanoparticle Hydrogel Enhances Potency and Breadth of Influenza Vaccination. Acs Biomater Sci Eng 7, 1889-1899 (2021).
35. Boopathy, A. V. et al. Enhancing humoral immunity via sustained-release implantable microneedle patch vaccination. Proc National Acad Sci 116, 16473-16478 (2019).
36. Gale, E. C. et al. Hydrogel-Based Slow Release of a Receptor-Binding Domain Subunit Vaccine Elicits Neutralizing Antibody Responses Against SARS-CoV-2. Adv Mater Deerfield Beach Fla 33, 2104362 (2021).
37. Demento, S. L. et al. Role of sustained antigen release from nanoparticle vaccines in shaping the T cell memory phenotype. Biomaterials 33, 4957-4964 (2012).
38. Hem, S. L. & HogenEsch, H. Relationship between physical and chemical properties of aluminum-containing adjuvants and immunopotentiation. Expert Rev Vaccines 6, 685-698 (2014).
39. Morefield, G. L. et ad. Role of aluminum-containing adjuvants in antigen internalization by dendritic cells in vitro. Vaccine 23, 1588-1595 (2005).
40. Jardine, J. et al Rational HIV immunogen Design to Target Specific Germline B Cell Receptors. Science 340, 711-716 (2013).

1. A composition comprising the general formula X1-X2-X3, wherein
(a) X2 comprises a antigen;
(b) X1 is absent or comprises or consists of the amino acid sequence (C)KKKTG (SEQ ID NO:1); and
(c) X3 is absent or comprises or consists of the amino acid sequence GTKKK(C) (SEQ ID NO:2),
wherein (i) only one of X1 and X3 is present, and (ii) the residue in parentheses in SEQ ID NO:1 and SEQ ID NO:2 is optional and may be present or may be deleted.

2. The composition of claim 1, wherein the antigen comprises a polypeptide antigen, and the composition comprises a fusion protein.

3. The fusion protein of claim 2, wherein optional amino acid linkers may be present between the X1-X2 or X2-X3.

4. The composition of claim 1, wherein X2 is present and consists of GTKKK(C) (SEQ ID NO:2) or GTKKK (SEQ ID NO:3).

5. The composition of claim 1, wherein X1 is present and consists of the amino acid sequence (C)KKKTG (SEQ ID NO:1) or KKKTG (SEQ ID NO:4).

6. The composition of claim 1, comprising the formula B1-X1-X2 or X2-X3-B2, wherein
B1 comprises pSer4-GGSGGGS-Z1 (SEQ ID NO:5), or
B2 comprises Z1-GGSGGGS-pSer4 (SEQ ID NO:6);
wherein Z1 comprises a linkage to the antigen.

7. The composition of claim 6, wherein Z1 is selected from the group consisting of maleimide, 5-hydroxy-pyrrolone, a 3-arylpropiolonitrile, and methylsulfonyl phenyloxadiazole.

8. The composition of claim 1, comprising:
(SEQ ID NO: 7)
X2-GTKKK-Z1-GGSGGGS-pSer4;
or
(SEQ ID NO: 8)
pSer4-GGSGGGS-Z1-KKKTG-X2.

9. The composition of claim 1, comprising:
(SEQ ID NO: 9)
X2-GTKKK-maleimide-GGSGGGS-pSer4;
or
(SEQ ID NO: 10)
pSer4-GGSGGGS-maelimide-KKKTG-X2.

10. The composition of claim 1, wherein the antigen comprises an antigen from a pathogen, such as a viral antigen or bacterial antigen, or a tumor antigen.

11. (canceled)

12. The fusion protein of claim 2, wherein the antigen forms a multimer, including but not limited to a dimer, trimer, tetramer, pentamer, or hexamer; preferably wherein the antigen forms a trimer

13. The fusion protein of claim 12, wherein the antigen comprises a human immunodeficiency virus (HIV) envelope (Env) antigen, an influenza hemagglutinin (HA) antigen, or an influenza neuraminidase (NA) antigen.

14. The fusion protein of claim 13, wherein the antigen comprises an amino acid sequence at least 70% identical to the amino acid sequence selected from the group consisting of SEQ ID NO:11-15, or an antigenic fragment thereof.

15. A polypeptide comprising a sequence selected from the group consisting of CGTKKK-Z1-GGSGGGS-pSer4 (SEQ ID NO:16) and pSer4-GGSGGGS-Z1-KKKTGC (SEQ ID NO:17), wherein Z1 comprises a linkage.

16. (canceled)

17. The composition of claim 6, bound to aluminum hydroxide via pSer4.

18. A formulation, comprising the composition of claim 17, further comprising a non-liposome, non-micelle particle, wherein the particle comprises a lipid, a sterol, a saponin, and an optional additional non-alum adjuvant, wherein the particle is optionally bound to the alum.

19.-37. (canceled)

38. A pharmaceutical composition comprising the composition of claim 1 and a pharmaceutically acceptable carrier, or a vaccine comprising the composition of claim 1.

39. (canceled)

40. A method for generating an immune response against an antigen or for treating a subject in need thereof, comprising administering to a subject an amount effective to generate an immune response in the subject of the composition of claim 1.

41.-43. (canceled)
